(19) United States
(12) Patent Application Publication
(10) Pub. No.: US 2022/0235348 A1
(43) Pub. Date: Jul. 28, 2022

(54) CRISPR METHODS FOR TREATING CANCERS

(71) Applicants: Board of Regents, The University of Texas System, Austin, TX (US); Tsinghua University, Beijing (CN)

(72) Inventors: Zhimin LU, Houston, TX (US); Ronald DEPINHO, Houston, TX (US); Dongming XING, Houston, TX (US)

(73) Assignees: Board of Regents, The University of Texas System, Austin, TX (US); Tsinghua University, Beijing (CN)

(21) Appl. No.: 17/611,470

(22) PCT Filed: May 15, 2020
(86) PCT No.: PCT/US20/33190

§ 371 Date: Nov. 15, 2021

Related U.S. Application Data
(60) Provisional application No. 62/848,347, filed on May 15, 2019.

(51) Int. Cl. C12N 9/78; A61P 35/00; C12N 9/22; C12N 15/11; C12N 15/86

(52) U.S. Cl. CPC C12N 9/78; A61P 35/00; C12N 9/22; C12N 15/11; C12N 15/86; C12N 2750/14171; C07K 2319/00; C07K 2319/09; C12N 2310/20; C12N 2750/14143; C12Y 305/04

(57) ABSTRACT

Methods for reversing one or more mutations in the telomerase (TERT) promoter are provided and may be used to treat a cancer. In some embodiments, programmable base editing (PBE) is used to correct a mutated TERT promoter (e.g., −124 C>T, −228C>T, or −250C>T to −124 C, −228C, or −250C, respectively) by using a single guide (sg) RNA-guided and deactivated Campylobacter jejuni Cas9-fused adenine base editor (CjABE). These methods can be used to treat a cancer, such as for example glioblastoma multiforme (GBM), in a mammalian subject in vivo.

This application claims the benefit of U.S. Provisional Patent Application No. 62/848,347, filed May 15, 2019, the entirety of which is incorporated herein by reference.

### BACKGROUND OF THE INVENTION

### 1. Field of the Invention

The present invention relates generally to the field of molecular biology and medicine. More particularly, it concerns CRISPR and CRISPRi based methods for treating cancers.

### 2. Description of Related Art

Cancer remains a particularly difficult clinical problem for many cancers. For example, glioblastoma multiforme (GBM) is particularly difficult to treat and has a median survival duration of only about 14 months (Cloughesy et al., 2014; Yuan et al., 2016). Telomerase (TERT) is active in many cancers, but TERT-targeted therapies have undergone limited development (Shay et al., 2016).

Clustered regularly interspaced short palindromic repeats (CRISPR) prokaryotic adaptive immune systems have been widely applied as tools for manipulating eukaryotic genomes. While it has been proposed that these methods may be used for therapeutic or clinical applications to treat a disease, the details regarding which approach might be used to treat which disease are still being evaluated. For example, while the potential of gene editing in correcting cancer-specific mutations has been suggested, the details regarding which mutations in which cancers may be amendable to alteration remains unclear and an area of intensive investigation. Clearly, there is a need for new methods for treating cancers.

### SUMMARY OF THE INVENTION

The present invention is based, in part, on the discovery that CRISPR methods such as nucleobase editors (NBE) can be effectively used to reverse one or more mutation in the telomerase (TERT) promoter (e.g., −124C>T, −228C>T, and/or −250C>T) to treat a disease such as a cancer. As shown in the examples, in contrast to other genes that are mutated and can give rise to or worsen a cancer prognosis, mutations in the TERT promoter were particularly susceptible to modifications using CRISPR-based methods and in particular were very susceptible to modification using nucleobase editors (NBEs). In contrast, NBEs such as use of sgRNA-guided Campylobacter jejuni Cas9 were dramatically less effective or not effective in modifying other cancer-causing or cancer-promoting mutations in other genes including K-Ras, B-Raf, PI3K, EGFR, IDH1/2, PTEN, BRAC1. These results support the idea that the effectiveness of CRISPR-based methods for reversing mutations found in cancers can vary highly based on the particular mutation targeted, and the TERT promoter may be particularly amenable to modification with a CRISPR-based method such as modification via an NBE. In some embodiments, a mutation in the TERT promoter (e.g., preferably a C>T point mutation such as −124C>T, −228C>T, or −250C>T) is reversed using sgRNA-guided Campylobacter jejuni Cas9. For example, local injection of AAVs expressing sgRNA-guided CjABE inhibited the growth of brain tumors with TERT promoter mutations, demonstrating higher therapeutic specificity for TERT promoter-mutated tumors than that of other approaches (such as use of small molecular compounds, short hairpin RNA, and small interfering RNA). It is anticipated that the methods provided herein may be used to treat a variety of cancers that have a TERT promoter mutation including, e.g., glioblastomas (GBMs), melanomas, urothelial carcinomas of the bladder, hepatocellular carcinomas, medulloblastomas, squamous cell carcinomas of the tongue, and thyroid cancers.

An aspect of the present invention relates to a method of treating a cancer in a mammalian subject comprising administering to the subject a CRIPSR therapy to reverse a point mutation in a telomerase reverse transcriptase (TERT) promoter in the cancer. In some preferred embodiments, the point mutation is a C>T point mutation (e.g., −124C>T, −228C>T, or −250C>T). The CRISPR therapy may comprise administering a nucleic acid encoding a sgRNA-guided Cas9 or nuclease-deactivated Cas9 (dCas9) to the subject. In some embodiments, the nucleic acid is delivered via a viral vector (e.g., an adenovirus, adeno-associated virus, retrovirus, lentivirus, Newcastle disease virus (NDV), or lymphocytic choriomeningitis virus (LCMV)). In some embodiments, the nucleic acid is delivered via an exosome, lipid-based transfection, nanoparticle, or cell-based delivery system. The CRISPR therapy may comprise administering a sgRNA-guided deactivated Cas9 that is fused to an adenine base editor (ABE) to the subject. In some embodiments, the deactivated Cas9 is a deactivated Campylobacter jejuni Cas9, S. pyogenes Cas9, or S. thermophiles Cas9. In some embodiments, the deactivated Cas9 is a deactivated Campylobacter jejuni Cas9. In some embodiments, the sgRNA-guided deactivated Cas9 that is fused to an adenine base editor (ABE) is further fused to a cell penetrating peptide (CPP) or nuclear localization signal. In some embodiments, the sgRNA-guided deactivated Cas9 that is fused to an adenine base editor (ABE) is delivered via a viral vector. In some embodiments, the adenine base editor comprises a mutation at one or more amino acid positions corresponding to amino acids that are involved in H-bond contacts with tRNA in a wild-type adenosine deaminase, preferably wherein the wild-type adenosine deaminase is a TadA deaminase. The TadA deaminase may comprise the mutations (A106V and D108N), or three or more of: W23R, H36L, (P48S or P48A), L84F, A106V, D108N, J123Y, S146C, D147Y, R152P, E155V, I156F, and/or K157N (e.g., A106V and D108N, plus 1, 2, 3, 4, 5, 6, 7, 8, 9, 10, or more of W23R, H36L, (P48S or P48A), L84F, J123Y, S146C, D147Y, R152P, E155V, I156F, and/or K157N). In some embodiments, the sgRNA-guided deactivated Cas9 and the adenine base editor (ABE) are separated by a linker. In some embodiments, the sgRNA-guided deactivated Cas9 that is fused to an adenine base editor (ABE) is further fused to a nuclear localization sequence (NLS), and/or an inhibitor of base repair, such as preferably a nuclease dead inosine specific nuclease (dISN). In some embodiments, the viral vector is an adenovirus, adeno-associated virus, retrovirus, lentivirus, Newcastle disease virus (NDV), or lymphocytic choriomeningitis virus (LCMV). In some embodiments, the sgRNA-guided deactivated Cas9 that is fused to an adenine base editor is delivered to the subject via an exosome, lipid-based transfection, nanoparticle, or cell-based delivery system. The CRISPR therapy may result in cancer-cell senescence or reduced proliferation of the cancer. The cancer may be a glioblastoma, glioma, melanoma, hepatocellular carcinoma, urothelial carcinoma, medulloblastoma, squamous cell carcinoma such as of the tongue or head and neck, brain cancer, thyroid cancer, adrenal cortical carcinoma, tumors of the female reproductive organs, such as ovarian carcinoma, uterine clear cell carcinoma, cervical squamous cell carcinoma, mantle cell lymphoma, fibrosarcoma, myxoid liposarcoma, meningioma, or renal cell carcinoma. In some embodiments, the cancer is a glioma, glioblastoma, or melanoma. The cancer may contain a mutation in one or more oncogenes. In some embodiments, the oncogene is K-Ras, B-Raf, EGFR, ALK, PI3K, BCR-ABL, IDHL or IDH2. In some embodiments, the subject is a human.

Another aspect of the present invention relates to a CRISPR therapy as described herein or above for use in treating a cancer in a mammalian subject, preferably a human. In some embodiments, the cancer is a glioblastoma, glioma, melanoma, hepatocellular carcinoma, urothelial carcinoma, medulloblastoma, squamous cell carcinoma such as of the tongue or head and neck, brain cancer, thyroid cancer, adrenal cortical carcinoma, tumor of the a female reproductive organ (e.g., an ovarian carcinoma, uterine clear cell carcinoma, or cervical squamous cell carcinoma), mantle cell lymphoma, fibrosarcoma, myxoid liposarcoma, meningioma, or renal cell carcinoma.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein. The patent or application file contains at least one drawing executed in color. Copies of this patent or patent application publication with color drawings(s) will be provided by the Office upon request and payment of the necessary fee.

FIGS. 1A-E: PBE of Mutated TERT Promoter Abrogates the Binding of ETS1 and GABPA to the Promoter. FIG. 1A, The DNA region spanning the mutation 1,295,113 C>T (−124 C>T) in the TERT promoter locus at chromosome 5 of the indicated cell lines was genotyped. Arrows indicate TERT promoter mutations and the WT TERT promoter. FIG. 1B, Diagram of HA-CjABE targeting the −124 C>T mutation under the guidance of a designed sgRNA expressed in an adeno-associated viral vector. HA-tagged CjABE was expressed under the control of the EF-1α core promoter. SgRNA targeting the TERT promoter mutation or a control sgRNA were expressed under the control of the U6 promoter. Both expression cassettes (EF-1α-HA-CjCas9 and U6-sgRNA) were inserted into an AAV type 2 vector and packaged into virions for cell infection. CjABE, which were expressed by AAVs, bound to the mutated TERT promoter and converted the targeted A base to I via deamination and, subsequently, to C via mismatch repair of tumor cells, leading to correction of the targeted T⋅A base pair to C⋅G in the mutant TERT promoter locus, thereby abrogating ETS-driven TERT transcription. FIG. 1C, The indicated cells were infected with the indicated AAVs expressing HA-CjABE under the guidance of sgRNA with or without targeting of the TERT promoter mutation at a multiplicity of infection (MOI) of 100. The time points of the AAV infection and DNA sequencing are shown in the upper panel. The DNA region spanning the mutation 1,295,113 C>T (−124 C>T) in the TERT promoter locus at chromosome 5 of the indicated cell lines was genotyped (lower panel). Arrows indicate the mutations. The nucleotide conversion rate was calculated by deduction of the indicated peak area compared to uncorrected peak area. FIG. 1D and FIG. 1E, The indicated cells were infected with the indicated AAVs expressing HA-CjABE under the guidance of sgRNA with or without targeting of the TERT promoter mutation at an MOI of 100 for 72 hr. ChIP analyses of the indicated cells were performed with anti-HA (FIG. 1D), anti-ETS1, and anti-GABPA (FIG. 1E) antibodies. The histogram shows the amount of immunoprecipitated DNA expressed as a percentage of the total input DNA. The depicted results are the averages from at least three independent experiments. Values are means±standard deviation (s.d.).

FIGS. 2A-C: PBE of the Mutated TERT Promoter Inhibits TERT Expression. FIG. 2A, A luciferase reporter assay was performed to measure the transcriptional activity of the TERT core promoter (−200 to +58 bp) with or without the −124 C>T mutation in the indicated cells, which are infected with AAVs (MOI=100) expressing HA-CjABE under the guidance of sgRNA with or without targeting of the TERT promoter mutation. The depicted results are the averages from at least three independent experiments. Values are means±s.d. FIG. 2B and FIG. 2C, The indicated cells were infected with AAVs expressing HA-CjABE under the guidance of sgRNAs with or without targeting of the TERT promoter mutation at an MOI of 100 for 72 hr. Quantitative polymerase chain reaction (PCR) analyses (FIG. 2B) and immunoblot analyses with the indicated antibodies (FIG. 2C) were performed.

FIGS. 3A-D: Mutated TERT Promoter-Targeted PBE Reduces Telomere Length and Induces Tumor-Cell Senescence and Proliferation Inhibition. FIG. 3A, QFISH analyses of telomere lengths in the indicated cells at the indicated time points after infection (as shown in FIG. 1C) with AAVs (MOI=100) expressing HA-CjABE under the guidance of sgRNA with or without targeting of the TERT promoter mutation (upper panels) were performed. The immunofluorescence intensity in 10 cells was quantitated using the ImageJ software program (lower panels). Values are means±s.d. FIG. 3B, TRF analyses were performed using the indicated cells at the indicated time points after infection (as shown in FIG. 1C) with AAVs (MOI=100) expressing HA-CjABE under the guidance of sgRNA with or without targeting of the TERT promoter mutation. FIG. 3C, The indicated cells were infected (as shown in FIG. 1C) with AAVs (MOI=100) expressing HA-CjABE under the guidance of sgRNAs with or without targeting of the TERT promoter mutation. Senescence-associated β-galactosidase stains of the indicated cells were performed 30 days after the first AAV infection. The percentages of β-galactosidase-positive cells are shown. The depicted results are the averages from at least three independent experiments. Values are means±s.d. FIG. 3D, The indicated cells were infected (as shown in FIG. 1C) with AAVs (MOI=100) expressing HA-CjABE under the guidance of sgRNAs with or without targeting of the TERT promoter mutation. Thirty days after the first AAV infection, 2×10⁵ U87 cells were plated and counted at the indicated time points. The depicted results are the averages from at least three independent experiments. Values are means±s.d.

FIGS. 4A-F. Mutated TERT Promoter-Targeted PBE Inhibits Brain Tumorigenesis. FIG. 4A, Flow chart of AAV injections into tumors. U87 cells with or without reconstituted expression of Flag-TERT were intracranially injected into athymic nude mice (n=8). AAVs expressing HA-CjABE under the guidance of sgRNAs with or without targeting of the TERT promoter mutation were injected into the brains of mice at the indicated time points after injection of U87 cells expressing luciferase. The frequencies of the virus injections and measurements of the luminescence of tumors luminescent measurements are shown. FIG. 4B, Luciferase-expressing U87 cells with or without reconstituted expression of Flag-TERT were intracranially injected into athymic nude mice (n=8). AAVs expressing HA-CjABE under the guidance of sgRNA with or without targeting of the TERT promoter mutation were delivered (as shown in FIG. 4A) into mice via intracranial injection. The luminescence intensity of tumor cells in representative mice at the indicated time points after cell injection is shown in the left panel. The bar graphs in the right panel show the relative luminescence intensity. Values are means±s.d. FIG. 4C, Luciferase-expressing U87 cells with or without reconstituted expression of Flag-TERT were intracranially injected into athymic nude mice (n=8). AAVs expressing HA-CjABE under the guidance of sgRNA with or without targeting of the TERT promoter mutation were delivered (as shown in FIG. 4A) into mice via intracranial injection. The survival times of the mice were recorded. FIG. 4D, Luciferase-expressing U87 cells with or without reconstituted expression of Flag-TERT were intracranially injected into athymic nude mice (n=8). AAVs expressing HA-CjABE under the guidance of sgRNA with or without targeting of the TERT promoter mutation were delivered (as shown in FIG. 4A) into the mice via intracranial injection. The mice were sacrificed 34 days after cell injection. Immunohistochemical analyses of the indicated brain tumor sections were performed with the indicated antibodies (left panel). TERT and Ki67 expression levels in tumor samples were quantified in 10 microscopic fields (right panel). FIG. 4E and FIG. 4F, Luciferase-expressing U87 cells with or without reconstituted expression of Flag-TERT were intracranially injected into athymic nude mice (n=8). AAVs expressing HA-CjABE under the guidance of sgRNA with or without targeting of the TERT promoter mutation were delivered (as shown in FIG. 4A, into the mice via intracranial injection. The mice were sacrificed 34 days after cell injection. TRF analyses of the indicated brain tumor tissues were performed (FIG. 4E). The anaphase bridge index in hematoxylin- and eosin-stained tumor sections was analyzed and calculated as the percentage of anaphases of mitotic cells with chromatin bridges. At least 30 anaphases of mitotic cells were examined per tumor sample. Arrows point to the cells in anaphase with chromatin bridges (FIG. 4F, left panel). The bar graphs show the anaphase bridge index in tumor sections (FIG. 4F, right panel). Values are means±s.d.

FIG. 5: Relative TERT mRNA levels.

FIG. 6A-G: PBE of the Mutated TERT Promoter Abrogates the Binding of ETS1 and GABPA to the Promoter. FIG. 6A, The DNA region spanning the mutation at chromosome 5, 1,295,113 C>T (−124 C>T) in the TERT promoter locus of the indicated cell lines was genotyped. Arrows indicate the mutation. FIG. 6B, The indicated cells were infected with AAVs expressing HA-CjABE under the guidance of sgRNAs with or without targeting of the TERTpromoter mutation at an MOI of 100 for 72 hr. Results of immunoblot analyses using the indicated antibodies are shown. WB, Western blot. FIG. 6C, The indicated cells were infected with AAVs expressing HA-CjABE under the guidance of sgRNAs with or without targeting of the TERTpromoter mutation at an MOI of 100 for 72 hr. The DNA region spanning the mutation at chromosome 5 (1,295,113 C>T [−124 C>T] in the TERT promoter locus) of the indicated cell lines was genotyped. Arrows indicate the TERT promoter mutation or the WT TERT promoter. FIG. 6D, The time points of the AAV infection and DNA sequencing were shown in FIG. 1C. The cells were harvested on day 10 after the first infection with AAVs expressing HA-CjABE under the guidance of sgRNAs with or without targeting of the TERT promoter mutation at MOI of 100. The DNA region spanning the mutation at chromosome 5 (1,295,113 C>T [−124 C>T] in the TERT promoter locus) of the indicated cell lines was genotyped. Arrows indicate the WT TERT promoter. FIG. 6E, The indicated cells were infected with AAVs expressing HA-dCjCas9 under the guidance of sgRNAs with or without targeting of the TERT promoter mutation at an MOI of 100 for 72 hr. Results of immunoblot analyses performed using the indicated antibodies are shown. FIG. 6F and FIG. 6G, The indicated cells were infected with AAVs expressing HA-dCjCas9 under the guidance of sgRNAs with or without targeting of the TERT promoter mutation at an MOI of 100 for 72 hr. Results of ChIP analyses of the indicated cells performed with anti-HA (FIG. 6F), anti-ETS1, and anti-GABPA (FIG. 6G) antibodies are shown. The histogram shows the amount of immunoprecipitated DNA expressed as a percentage of the total input DNA. The depicted results are the averages from at least three independent experiments. Values are means±s.d.

FIGS. 7A-B: Mutated TERT Promoter-Targeted PBE Does Not Affect Telomere Length in LN18 and SVG Cells. FIG. 7A, QFISH analyses of telomere (Tel) lengths were performed using the indicated cells at the indicated time points after repeated infection (as shown in FIG. 1C) with AAVs (MOI=100) expressing HA-CjABE under the guidance of sgRNA with or without targeting of the TERT promoter mutation (left panels). The immunofluorescence intensity in 10 cells was quantitated using the ImageJ software program (right panels). Values are means±s.d. DAPI, 4′,6-diamidino-2-phenylindole. FIG. 7B, TRF analyses were performed using the indicated cells at the indicated time points after repeated infection (as shown in FIG. 1C) with AAVs (MOI=100) expressing HA-CjABE under the guidance of sgRNA with or without targeting of the TERT promoter mutation.

FIGS. 8A-C: PBE Corrects the TERT Promoter Mutation in U87 Cells and Tumors Derived from U87 Cells. FIG. 8A and FIG. 8B, Luciferase-expressing U87 cells were infected with AAVs (MOI=100; as shown in FIG. 1C) expressing HA-CjABE under the guidance of sgRNA targeting the TERT promoter mutation. These cells were stably transfected with a vector expressing Flag-TERT. Results of immunoblot analyses with the indicated antibodies (FIG. 8A) and genotyping of the DNA region spanning the mutation of at chromosome 5 (1,295,113 C>T [−124 C>T] in the TERT promoter locus) (FIG. 8B) are shown. Arrows indicate the mutations. WB, Western blot. FIG. 8C, Luciferase-expressing U87 cells with or without reconstituted expression of Flag-TERT proteins were intracranially injected into athymic nude mice (n=8). AAVs expressing HA-CjABE under the guidance of sgRNA with or without targeting of the TERT promoter mutation were delivered (as shown in FIG. 4A) to the mice via intracranial injection. The mice were sacrificed 34 days after cell injection. Genotype analyses of the DNA region spanning the mutation at chromosome 5 (1,295,113 C>T [−124 C>T] in the TERT promoter locus) in the indicated brain tumors were performed. Arrows indicate the mutations.

### DESCRIPTION OF ILLUSTRATIVE EMBODIMENTS

In some aspects, methods for reversing a mutation in a TERT promoter to treat a cancer are provided. For example, as shown in the below examples, CRISPR interference and PBE were tested to determine their potential in editing a TERT gene promoter-activating mutation, which occurs in many diverse cancer types, such as glioblastoma multiforme (GBM). The correction of the mutated TERT promoter −124 C>T to −124 C was achieved using a single guide (sg) RNA-guided and deactivated Campylobacter jejuni Cas9-fused adenine base editor (CjABE). This modification blocked E-twenty-six transcription factor family members' binding to the TERT promoter, reduced TERT transcription and TERT protein expression, and induced cancer-cell senescence and proliferative arrest. These approaches may thus be used to correct other TERT promoter mutations such as, e.g., −228C>T and/or −250C>T. Local injection of adeno-associated viruses expressing sgRNA-guided CjABE inhibited the growth of gliomas harboring TERT promoter mutations. These data indicate that these gene editing approach can be used to treat a cancer, and the data also validates activated TERT promoter mutations as a cancer-specific therapeutic target.

### I. Definitions

The term “deaminase” or “deaminase domain” refers to a protein or enzyme that catalyzes a deamination reaction. In some embodiments, the deaminase is an adenosine deaminase, which catalyzes the hydrolytic deamination of adenine or adenosine. In some embodiments, the deaminase or deaminase domain is an adenosine deaminase, which can catalyze the hydrolytic deamination of adenosine or deoxyadenosine to inosine or deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA deaminase (ecTadA). In some embodiments, the TadA deaminase is a truncated E. coli TadA deaminase. For example, the truncated ecTadA may be missing one or more N-terminal amino acids relative to a full-length ecTadA. In some embodiments, the truncated ecTadA may be missing 1, 2, 3, 4, 5, 6, 7, 8, 9, 10, 11, 12, 13, 14, 15, 6, 17, 18, 19, or 20 N-terminal amino acid residues relative to the full length ecTadA. In some embodiments, the ecTadA deaminase does not comprise an N-terminal methionine.

The term “base editor (BE),” or “nucleobase editor (NBE)” refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating an adenine (A) in DNA. In some embodiments, the base editor is a fusion protein comprising a nucleic acid programmable DNA binding protein (napDNAbp) fused to an adenosine deaminase. In some embodiments, the base editor is a Cas9 protein fused to an adenosine deaminase. In some embodiments, the base editor is a Cas9 nickase (nCas9) fused to an adenosine deaminase. In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In some embodiments, the dCas9 domain of the fusion protein comprises a D10A and a H840A mutation of Cas9 from Streptococcus pyogenes (NCBI Reference Sequence: NC 002737.2), or a corresponding mutation in another Cas9 protein, which inactivates the nuclease activity of the Cas9 protein. In some embodiments, the fusion protein comprises a D10A mutation and comprises a histidine at residue 840 of Cas9 from Streptococcus pyogenes, or another Cas9 protein, which renders Cas9 capable of cleaving only one strand of a nucleic acid duplex. Examples of a Cas9 nickase or catalytically inactive Cas9 proteins can be found, e.g., in US 2019/0093099.

The term “linker,” as used herein, refers to a bond (e.g., covalent bond), chemical group, or a molecule linking two molecules or moieties, e.g., two domains of a fusion protein, such as, for example, a nuclease-inactive Cas9 domain and a nucleic acid-editing domain (e.g., an adenosine deaminase). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated. In some embodiments, a linker comprises the amino acid sequence SGSETPGTSESATPES (SEQ ID NO: 2), which may also be referred to as the XTEN linker. In some embodiments, a linker comprises the amino acid sequence SGGS (SEQ ID NO: 3). In some embodiments, a linker comprises (SGGS)ₙ (SEQ ID NO: 3), (GGGS)ₙ (SEQ ID NO: 4), (GGGGS)ₙ (SEQ ID NO: 5), (G)ₙ, (EAAAK)ₙ (SEQ ID NO: 6), (GGS)ₙ, SGSETPGTSESATPES (SEQ ID NO: 2), or (XP)ₙ motif, or a combination of any of these, wherein n is independently an integer between 1 and 30, and wherein X is any amino acid. In some embodiments, n is 1, 2, 3, 4, 5, 6, 7, 8, 9, 10, 11, 12, 13, 14, or 15.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y., 2012).

The term “inhibitor of base repair” or “IBR” refers to a protein that is capable in inhibiting the activity of a nucleic acid repair enzyme, for example a base excision repair enzyme. In some embodiments, the IBR is an inhibitor of inosine base excision repair. Exemplary inhibitors of base repair include inhibitors of APE1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is a catalytically inactive EndoV or a catalytically inactive hAAG.

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published as WO/2001/038547 on May 31, 2001, the contents of which are incorporated herein by reference for their disclosure of exemplary nuclear localization sequences. In some embodiments, an NLS comprises the amino acid sequence PKKKRKV (SEQ ID NO: 7) or

(SEQ ID NO: 8)
MDSLLMNRRKFLYQFKNVRWAKGRRETYLC.

The term “nucleic acid programmable DNA binding protein” or “napDNAbp” refers to a protein that associates with a nucleic acid (e.g., DNA or RNA), such as a guide nucleic acid, that guides the napDNAbp to a specific nucleic acid sequence. For example, a Cas9 protein can associate with a guide RNA that guides the Cas9 protein to a specific DNA sequence that has complementary to the guide RNA. In some embodiments, the napDNAbp is a class 2 microbial CRISPR-Cas effector. In some embodiments, the napDNAbp is a Cas9 domain, for example a nuclease active Cas9, a Cas9 nickase (nCas9), or a nuclease inactive Cas9 (dCas9). Examples of nucleic acid programmable DNA binding proteins include, without limitation, Cas9 (e.g., dCas9 and nCas9), CasX, CasY, Cpf1, C2c1, C2c2, C2C3, and Argonaute. It should be appreciated, however, that nucleic acid programmable DNAbinding proteins also include nucleic acid programmable proteins that bind RNA. For example, the napDNAbp may be associated with a nucleic acid that guides the napDNAbp to an RNA. Other nucleic acid programmable DNA binding proteins are also within the scope of this disclosure, though they may not be specifically listed in this disclosure.

The term “Cas9” or “Cas9 domain” refers to an RNA-guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek et al., 2012, the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek et al., 2012, the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski et al., “The tracrRNA and Cas9 families of type II CRISPR-Cas immunity systems,” RNA Biology 10:5, 726-737, 2013; the entire contents of which are incorporated herein by reference. In some embodiments, a Cas9 nuclease has an inactive (e.g., an inactivated) DNA cleavage domain, that is, the Cas9 is a nickase. In some preferred embodiments, the Cas9 is from Campylobacter jejuni.

A nuclease-inactivated Cas9 protein may interchangeably be referred to as a “dCas9” protein (for nuclease-“dead” Cas9). Methods for generating a Cas9 protein (or a fragment thereof) having an inactive DNA cleavage domain are known (See, e.g., Jinek et al., 2012); Qi et al., 2013, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek; Qi et al., 2013). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

The term “RNA-programmable nuclease,” and “RNA-guided nuclease” are used interchangeably herein and refer to a nuclease that forms a complex with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., 2012, the entire contents of which is incorporated herein by reference. Other examples of gRNAs (e.g., those including domain 2) can be found in U.S. Provisional patent application, U.S. Ser. No. 61/874,682, filed Sep. 6, 2013, entitled “Switchable Cas9 Nucleases And Uses Thereof,” and U.S. Provisional patent application, U.S. Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example, Cas9 (Csn1) from Streptococcus pyogenes (see, e.g., “Complete genome sequence of an M1 strain of Streptococcus pyogenes.” Ferretti et al., 2001; “CRISPR RNA maturation by trans-encoded small RNA and host factor RNase III.” Deltcheva et al., Nature 471:602-607, 2011; and “A programmable dual-RNA-guided DNA endonuclease in adaptive bacterial immunity.” Jinek et al., 2012, the entire contents of each of which are incorporated herein by reference.

The term “effective amount,” as used herein, refers to an amount of a biologically active agent that is sufficient to elicit a desired biological response. For example, in some embodiments, an effective amount of a nucleobase editor may refer to the amount of the nucleobase editor that is sufficient to induce mutation of a target site specifically bound mutated by the nucleobase editor. In some embodiments, an effective amount of a fusion protein comprising a nucleic acid programmable DNA binding protein and a deaminase domain (e.g., an adenosine deaminase domain) can refer to the amount of the fusion protein that is sufficient to induce editing of a target site specifically bound and edited by the fusion protein. As will be appreciated by the skilled artisan, the effective amount of an agent, e.g., a fusion protein, a nucleobase editor, a deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. The term “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. The terms “nucleic acid,” “DNA,” and “RNA,” include nucleic acid analogs, e.g., analogs having other than a phosphodiester backbone. Nucleic acids can be purified from natural sources, produced using recombinant expression systems and optionally purified, or chemically synthesized, etc., as would be understood by one of skill in the art. Nucleic acids can comprise nucleoside analogs such as analogs having chemically modified bases or sugars, and/or backbone modifications.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “target site” refers to a sequence within a nucleic acid molecule that is deaminated by a deaminase or a fusion protein comprising a deaminase, (e.g., a dCas9-adenosine deaminase fusion protein provided herein).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, such as a cancer. In some embodiments, treatment may be administered after one or more symptoms have developed and/or after a disease has been diagnosed, such as after the diagnosis of a cancer expressing a TERT promoter mutation. Treatment may also be continued after symptoms have resolved, for example, to prevent or delay their recurrence.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

### II. CRISPR Methods

CRISPR prokaryotic adaptive immune systems have been used as tools for manipulating eukaryotic genomes. For example, the CRISPR-associated Cas9 protein from Streptococcus pyogenes or Campylobacter jejuni (Cj), together with a chimeric single guide (sg) RNA, is a programmable endonuclease that can be used to modify, regulate, or mark genomic loci in a wide variety of cells and organisms (Doudna et al., 2014). In addition, further modified catalytically deactivated Cas9, which does not cleave the target gene and cause CRISPR interference (CRISPRi), is a programmable DNA-binding protein that can turn targeted genes on and off, mark specific genomic loci with fluorescent proteins, and alter epigenetic marks with no apparent off-target effects (Doudna et al., 2014; Qi et al. 2013; Maeder et al. 2013; Gilbert et al. 2013). Programmable base editing (PBE) with an adenine base editor (ABE) composed of a fused transfer RNA adenosine deaminase and deactivated Cas9 converts targeted A⋅T base pairs efficiently to G⋅C base pairs with high product purity and low rates of undesired products, such as stochastic insertions and deletions (indels). ABEs can introduce point mutations more efficiently and cleanly than do a current Cas9 nuclease-based method (CORRECT) without double-stranded DNA cleavage and with fewer off-target genome modifications (Gaudelli et al., 2017).

In some aspects, a CRISPR method such as programmable base editing (PBE) may be used to reverse a mutation in the TERT promoter in a cancer in a mammalian subject. The TERT gene encodes for a highly specialized reverse transcriptase that adds hexamer repeats to the 3′ ends of chromosomes (Cesare et al., 2010; Aubert et al., 2008). Although somatic mutations in the TERT coding region are not common in human tumors, germline and somatic mutations of the TERT promoter are present in high percentages in many human cancers, including gliomas (83% of primary glioblastomas [GBMs], the most common primary brain tumor type), melanomas (71%), urothelial carcinomas of the bladder (66%), hepatocellular carcinomas (59%), medulloblastomas, squamous cell carcinomas of the tongue, and thyroid cancers (Horn et al. 2013; Huang et al., 2013; Killela et al., 2013; Nault et al., 2013; Kinde et al., 2013). Such mutations have occurred in two hotspot positions located −124 and −146 bp upstream of the ATG start site (−124 G>A and −146 G>A, respectively; −124 C>T and −146 C>T on the opposite strand). These mutations generate a de novo consensus binding site (GGAA) within the TERT promoter region for E-twenty-six (ETS) transcription factor family members, including ETS1 and the multimeric GA-binding protein A (GABPA), and confer increased TERT promoter activity (Horn et al. 2013; Huang et al., 2013; Bell et al. 2015; Stern et al. 2015). Reverting TERT promoter mutations or creating these mutations using the CRISPR-Cas9 approach demonstrated that these mutations are critical for increased telomerase promoter activity (Chiba et al., 2015; Xi et al., 2015; Li et al., 2015). In cancer cells, this increased telomerase promoter activity leads to enhanced expression of TERT and preservation of telomeres, enabling tumor cells to proliferate and evade senescence (Cesare et al., 2010). As shown in the below examples in both in vitro and in vivo experiments, expression of an sgRNA-guided deactivated CjCas9 (dCjCas9)-fused ABE (CjABE) converted the mutated TERT promoter −124 C>T to −124 C, blocked ETS binding to the TERT promoter, reduced TERT transcription and TERT protein expression, induced tumor-cell senescence and proliferative arrest, and inhibited brain tumor growth.

CRISPR methods that may be used herein include programmable base editing (PBE) and CRISPR interference (CRISPRi). As would be appreciated by one of skill, CRISPR methods generally rely on use of a single-stranded guide RNA (gRNA) and a CRISPR-associated (Cas) nuclease to cause a double-strand break in a specific DNA sequence. The guide RNA is a specific RNA sequence that recognizes the target DNA region of interest and directs the Cas nuclease there for editing. The gRNA is made up of two parts: crispr RNA (crRNA), a 17-20 nucleotide sequence complementary to the target DNA, and a tracr RNA, which serves as a binding scaffold for the Cas nuclease. A variety of CRISPR methods are known, including those described in US2015356239, US2015356239, WO2015089351, WO2015106004, US2013130248, WO2015157534, US2015218573, WO2015200555, and US20150376587.

CRISPRi can inhibit gene expression by using a catalytically dead Cas9 (dCas9) protein that lacks endonuclease activity to regulate genes in an RNA-guided manner. Targeting specificity can be determined by complementary base-pairing of a single guide RNA (sgRNA) to the genomic loci. The sgRNA is a chimeric noncoding RNA that can be subdivided into three regions: a 20 nt base-pairing sequence, a 42 nt dCas9-binding hairpin and a 40 nt terminator. Once the sgRNA selectively binds a DNA sequence and recruits the dCas9, transcriptional repression may occur due to steric hindrance preventing an RNA polymerase from initiating transcription or by disrupting elongation of the generation of a mRNA transcript by the RNA polymerase. Methods of CRISPRi are known in the art, e.g., as described in Qi et al. (2013).

A. Programmable Base Editing

Programmable base editing (PBE), such as adenine base editors (ABEs), provide a method for inducing single nucleotide changes in DNA with high fidelity and low off-target mutations. Base editing is a form of genome editing that enables direct, irreversible conversion of one base pair to another at a target genomic locus without requiring double-stranded DNA breaks (DSBs), homology-directed repair (HDR) processes, or donor DNA templates. Compared with standard genome editing methods to introduce point mutations, base editing can proceed more efficiently, and with far fewer undesired products such as stochastic insertions or deletions (indels) or translocations.

A variety of base editors are known and may can be used in some embodiments. For example, third-generation base editors designs (BE3) have been used and generally comprise: (i) a catalytically impaired CRISPR-Cas9 mutant that cannot make DSBs, (ii) a single-strand specific cytidine deaminase that converts C to uracil (U) within a ˜5-nucleotide window in the single-stranded DNA bubble created by Cas9, (iii) a uracil glycosylase inhibitor (UGI) that impedes uracil excision and downstream processes that decrease base editing efficiency and product purity, and (iv) nickase activity to nick the non-edited DNA strand, directing cellular DNA repair processes to replace the G-containing DNA strand. Together, these components have been shown to be able to cause permanent C⋅G to T⋅A base pair conversion in a variety of cells and organisms, including: bacteria, yeast, plants, zebrafish, mammalian cells, mice, and human cells. Base editing approaches can benefit from base editors that include protospacer-adjacent motif (PAM) compatibilities, narrowed editing windows, enhanced DNA specificity, and small-molecule dependence. Fourth-generation base editors (BE4 and BE4-Gam) have been used to further improve editing efficiency and product purity. Fourth-generation base editors are described, e.g., in Komor et al., 2017.

Later generation base editors are used in some preferred embodiments. Protein evolution and engineering has been used to generate adenine base editors (ABEs) that can convert A⋅T to G⋅C base pairs in DNA in bacteria and human cells. Seventh-generation ABEs efficiently convert A⋅T to G⋅C at a wide range of target genomic loci in human cells efficiently and with a very high degree of product purity, exceeding the typical performance characteristics of BE3. ABEs greatly expand the scope of base editing and, together with previously described base editors, enable programmable installation of all four transitions (C to T, A to G, T to C, and G to A) in genomic DNA. In some preferred embodiments, ABEs are used that can convert A-T base pairs to G-C base pairs, for examples such as those disclosed in Gaudelli et al., 2017.

For example, in some embodiments, the ABE is a fusion protein comprising a Cas9 (e.g., a Cas9 nickase or nCas9) domain and an adenosine deaminase that can deaminate adenosine in DNA. The adenine deaminase may be an E. coli TadA, human ADAR2, mouse ADA, or human ADAT2. The adenine deaminase may comprise one or more mutations; for example, the adenine deaminase may be a E. coli TadA (ecTadA) comprising at least (A106V and D108N), or more preferably three or more of W23R, H36L, (P48S or P48A), L84F, A106V, D108N, J123Y, S146C, D147Y, R152P, E155V, I156F, and/or K157N. In some embodiments, the TadA is a S. aureus TadA mutant. The TadA portion of the fusion protein may be a truncation of a full length ecTadA protein, such as the N-terminal truncations of ecTadA or an ecTadA mutant of SEQ ID NO:1 or as described in U.S. 2019/0093099. The Cas9 (e.g., Cas9 nickase) and the adenosine deaminase may be separated by a linker, such as a 32 amino acid linker (SGGS)₂XTEN-(SGGS)₂ (SEQ ID NO: 9). In some embodiments, the fusion proteins further comprise a nuclear localization sequence (NLS), and/or an inhibitor of base repair, such as, a nuclease dead inosine specific nuclease (dISN). In some embodiments, the Cas9 is a Campylobacter jejuni Cas9.

In some embodiments, the NBE may contain two ecTadA domains and a nucleic acid programmable DNA binding protein (napDNAbp). For example, the NBE may have the general structure ecTadA(D108N)-ecTadA(D108N)-nCas9. In some embodiments, an NBE containing a mutant ecTadA variants provided can be used to increase modification of nucleobase editing in mammalian cells. The Cas9 domain of the fusion protein may be a nuclease dead Cas9 (dCas9), a Cas9 nickase (nCas9), or a nuclease active Cas9. In some embodiments, the Cas9 is a Campylobacter jejuni Cas9. The fusion protein may further comprise an inhibitor of inosine base excision repair, for example a dISN or a single stranded DNA binding protein. Additional NBEs that may be used in various embodiments of the present invention include those described in US 2019/0093099.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and H840A mutations of Cas9 from Streptococcus pyogenes (NCBI Reference Sequence: NC_002737.2) or corresponding mutations in another Cas9. In some preferred embodiments, the dCas9 is derived from Campylobacter jejuni.

Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC 021314.1); Belliella baltica (NCBI Ref: NC_018010.1); Psychroflexus torquis (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP 002342100.1) or to a Cas9 from any other organism.

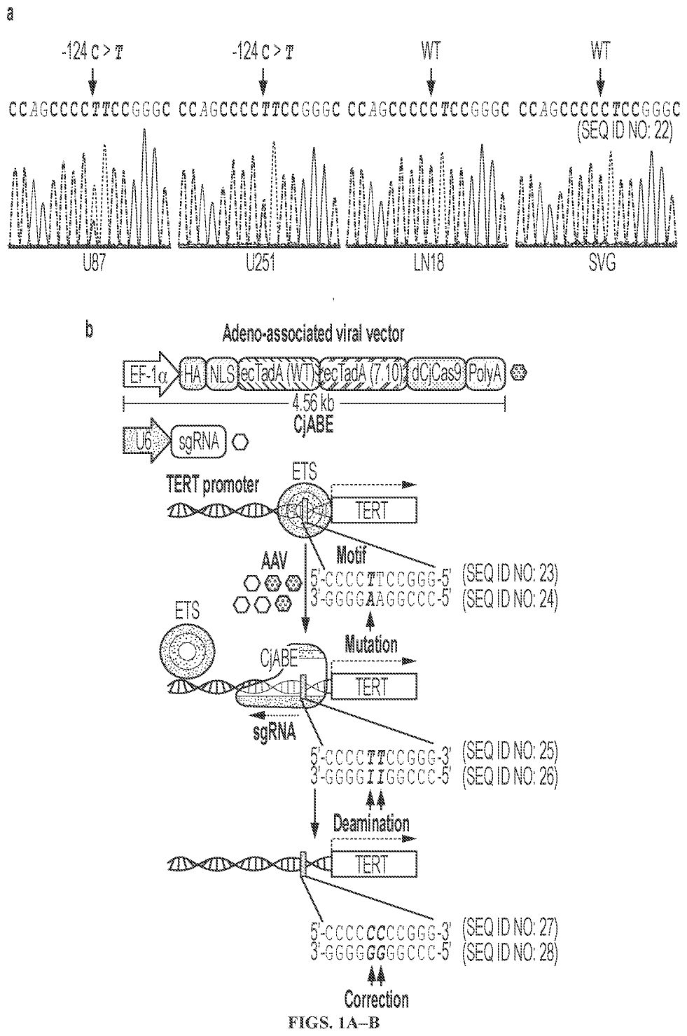

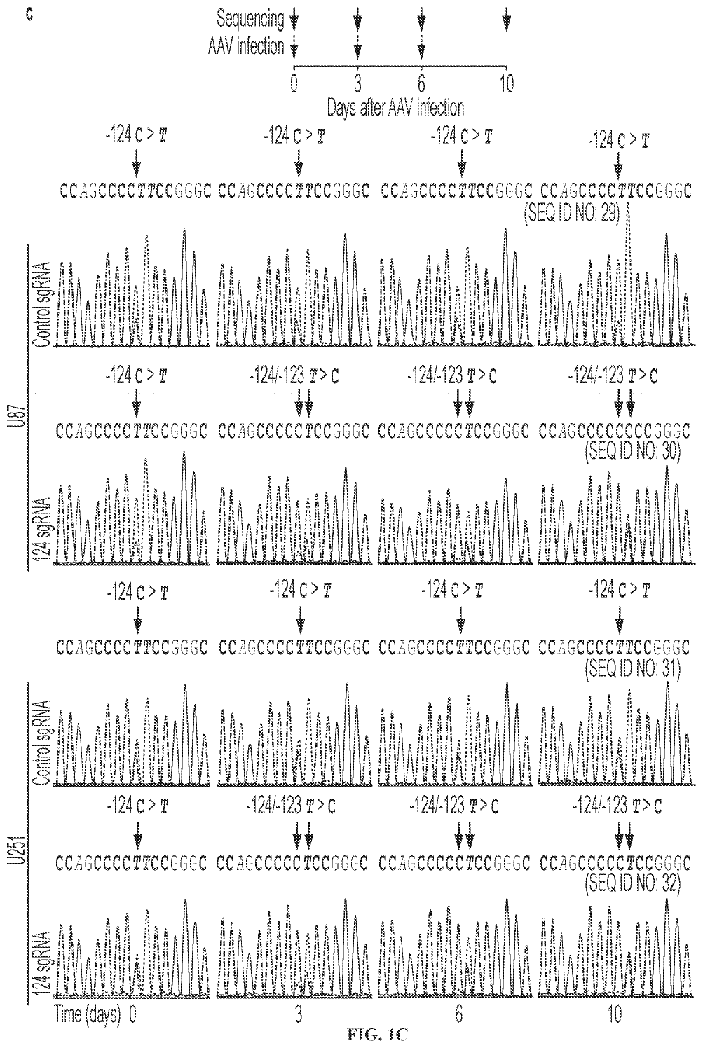

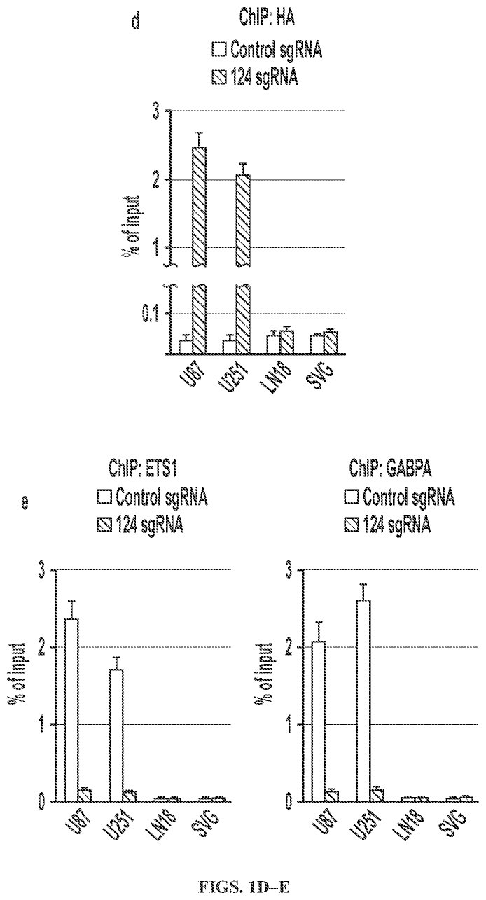

In some embodiments, a TERT promoter mutation can be reversed in a cancer in a mammalian subject, using the following methods. An sgRNA may be designed with complementary sequences covering −124 C>T, which is nine nucleotides away from the protospacer-adjacent motif (PAM) 5′-GGAAACC-3′ spanning −136 to −142 bp in the TERT promoter region (Yamada et al. 2017). An adeno-associated virus (AAV) type 2 vector (Swiech et al., 2015) can be constructed to express a hemagglutinin (HA)-tagged deactivated CjCas9-fused ABE protein (CjABE) containing a nuclear localization sequence, wild-type (WT) Escherichia coli tRNA-specific adenosine deaminase (ecTadA), evolved ecTadA (version 7.10), and dCjCas9 protein (e.g., as shown in FIG. 1B, or in Gaudelli et al., 2017). The vector may also express the sgRNA targeting the TERT −124 C>T mutation or a nontargeting sgRNA.

### III. Vectors and Viral Delivery

In some embodiments, the CRISPR therapy or PGE is delivered to a mammalian subject via viral delivery. For example, a virus may comprise a nucleic acid or vector that encodes a Cas9-fused ABE protein (e.g., a CjCas9-fused ABE protein (CjABE) containing a nuclear localization sequence) and a sgRNA. A variety of viruses are known in the art and may be used in various embodiments to deliver a CRISPR therapy, such as CRISPRi or PGE, to a mammalian subject. For example, the vector may be a viral expression vector such as, e.g., an adenovirus, adeno-associated virus, a retrograde virus, retrovirus, herpesvirus, lentivirus, poxvirus or papiloma virus expression vector.

One of skill in the art would be well-equipped to construct the vector through standard recombinant techniques (e.g., Sambrook et al., 2001). Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors (e.g., an AAV2/1 vector), retrograde AAV vectors, CAV vectors, rabies and pseudorabies vectors, herpes virus vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, and Rous sarcoma virus vectors.

In some embodiments, the virus is a retrovirus. Retroviruses have promise as gene delivery vectors due to their ability to integrate their genes into the host genome, transfer a large amount of foreign genetic material, infect a broad spectrum of species and cell types, and be packaged in special cell-lines. To construct a retroviral vector, a nucleic acid can be inserted into the viral genome in place of certain viral sequences to produce a virus that is replication-defective. To produce virions, a packaging cell line containing the gag, pol, and env genes—but without the LTR and packaging components—can be constructed. When a recombinant plasmid containing a cDNA, together with the retroviral LTR and packaging sequences, is introduced into a special cell line (e.g., by calcium phosphate precipitation), the packaging sequence allows the RNA transcript of the recombinant plasmid to be packaged into viral particles, which are then secreted into the culture medium. The medium containing the recombinant retroviruses can be collected, optionally concentrated, and used for gene transfer. Retroviral vectors are able to infect a broad variety of cell types. However, integration and stable expression typically require the division of host cells.

Lentiviruses are complex retroviruses, which, in addition to the common retroviral genes gag, pol, and env, contain other genes with regulatory or structural function. Lentiviral vectors are well known in the art (e.g., U.S. Pat. Nos. 6,013,516 and 5,994,136).

Recombinant lentiviral vectors are generally capable of infecting non-dividing cells and can be used for both in vivo and ex vivo gene transfer and expression of nucleic acid sequences. For example, recombinant lentivirus can infect a non-dividing cell—wherein a suitable host cell is transfected with two or more vectors carrying the packaging functions, namely gag, pol and env, as well as rev and tat—as described for example in U.S. Pat. No. 5,994,136.

In some embodiments, an episomal vector is used. The episomal vector may, e.g., be a plasmid- or liposome-based extra-chromosomal (i.e., episomal) vector. Episomal vectors include, e.g., oriP-based vectors, and/or vectors encoding a derivative of EBNA-1. These vectors may permit large fragments of DNA to be introduced unto a cell and maintained extra-chromosomally, replicated once per cell cycle, partitioned to daughter cells efficiently, and elicit reduced or substantially no immune response. In some embodiments, the episomal vector is derived from a rabies virus, a chicken anaemia virus (CAV virus), pseudorabies, or an AAV virus modified for retrograde transfer.

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Herpes simplex virus (HSV) is not a “lymphotrophic” herpes virus. Exemplary lymphotrophic herpes viruses include, but are not limited to EBV, Kaposi's sarcoma herpes virus (KSHV); Herpes virus saimiri (HS) and Marek's disease virus (MDV). Other sources of episome-base vectors are also contemplated, such as yeast ARS, adenovirus, SV40, or BPV.

In some embodiments, the delivery of the CRISPR therapy can use a transposon-transposase system. For example, the transposon-transposase systems that may be used include Sleeping Beauty, the Frog Prince transposon-transposase system (e.g., EP1507865), or the TTAA-specific transposon PiggyBac system. Generally, transposons are sequences of DNA that can move around to different positions within the genome of a single cell, a process called transposition. In the process, they can cause mutations and change the amount of DNA in the genome. Transposons were also once called jumping genes, and are examples of mobile genetic elements.

### IV. Pharmaceutical Compositions

Introduction of a nucleic acid encoding a CRISPR therapy such as CRISPRi or a PGE, into the host cells may use any suitable methods for nucleic acid delivery for transformation of a cell, as described herein or as would be known to one of ordinary skill in the art. In some embodiments, the CRISPR therapy is administered to a mammalian subject to treat a cancer.

Pharmaceutical compositions of the present invention comprise an effective amount of one or more compounds of the present invention, e.g., a CRISPR therapy (e.g., vector encoding a PBE to reverse a mutation in the TERT promoter), or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one compound or CRISPR therapy or additional active ingredient will be known to those of skill in the art in light of the present disclosure, as exemplified by Remington: The Science and Practice of Pharmacy, 21^{st} Ed., Lippincott Williams and Wilkins, 2005, incorporated herein by reference. Moreover, for animal (e.g., human) administration, it will be understood that preparations should typically meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (e.g., Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The a CRISPR therapy (e.g., a nucleic acid encoding a PBE to reverse a mutation in the TERT promoter, optionally comprised in a viral vector) may comprise different types of carriers depending on the route of administration (e.g., injection). The CRISPR therapy can be administered intravenously, intradermally, intracranially, transdermally, intrathecally, intraarterially, intraperitoneally, intramuscularly, intratumorally, subcutaneously, mucosally, locally, inhalation (e.g., aerosol inhalation), via injection, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference). The CRISPR therapy may be comprised, e.g., in liposomes, nanoparticles, an adenovirus or adeno-associated virus, a retrovirus, membrane derived vesicles, nanoformulations, or exosomes (e.g., as described in Biagioni et al. J Biol Eng. 2018; 12: 33; or Lino et al. Drug Deliv. 2018 November; 25(1):1234-1257).

In some embodiments, the CRISPR therapy (e.g., a nucleic acid encoding a PBE to reverse a mutation in the TERT promoter) is administered in nanoparticles or liposomes. Liposomes are well known in the art and include cationic and neutral liposomes. For example, liposomes can be unilamellar, multilamellar, or multivesicular. Additional varieties of liposomes and nanoparticles are known and may be used in various embodiments. For example, exosome-liposome hybrid nanoparticles may be used to deliver the CRISPR therapy (e.g., as described in Lin et al. Adv Sci (Weinh). 2018 April; 5(4): 1700611). In some embodiments, liposome-templated hydrogel nanoparticles can be used to deliver the CRISPR therapy (e.g., Biagioni et al. J Biol Eng. 2018; 12: 33.).

In some embodiments, a CRISPR therapy as disclosed herein is administered to a subject in combination (e.g., before, after, or substantially concurrently) with a second anti-cancer therapy to a mammalian subject, such as a human. The second anti-cancer therapy can be, e.g., a chemotherapy, a radiotherapy, an immunotherapy, a checkpoint inhibitor, a cell therapy, a gene therapy, or a surgery. For example, it is anticipated that the methods provided herein may be used in combination with a wide variety of chemotherapeutics.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Inhibition of Cancers In Vitro

A plasmid expressing dCjCas9 was generated by introducing mutations of D8A and H559A into WT CjCas9 in a PX404 plasmid (Catalog number: 68338; Addgene, Cambridge, Mass.) using a QuikChange site-directed mutagenesis kit (Stratagene, La Jolla, Calif.) and was designated as PX404d (Yamada et al., 2017). The DNA sequence encoding HA-tagged WT ecTadA, TadA (7.10), and the N-terminus (1-166 amino acids) of dCjCas9 were synthesized by Thermo Fisher Scientific. An AgeI/PflMI-digested fragment containing the HA-ecTadA (WT)-ecTadA (7.10)-dCjCas9 (1-166 amino acids) cassette was ligated into AgeI/PflMI-digested PX404d, and the construct was designated as PX404TadA. The open reading frame of HA-ecTadA (WT)-ecTadA (7.10)-dCjCas9 was amplified from PX404TadA using high-fidelity PCR and then ligated into the AgeI/NotI-digested adeno-associated viral vector pAAV-EFS-SpCas9 (Catalog number: 200932; Addgene), and the construct was designated as pAAV-CjABE. To construct the adeno-associated viral vector expressing sgRNA for CjCas9, annealed and phosphorylated sgRNA backbone oligonucleotides (forward, 5′-CTTCTGTTTTAGTCCCTGAAGGGACTAAAATAAAGAGTTTGCGGGACTCTGCGGG GTTACAATCCCCTAAAACCGCTTTTTTTCTAGACTGCAGAGGGCC-3′ (SEQ ID NO: 10); reverse, 5′-CTCTGCAGTCTAGAAAAAAAGCGGTTTTAGGGGATTGTAACCCCGCAGAGTCCC GCAAACTCTTTATTTTAGTCCCTTCAGGGACTAAAACAGAAGAGCT-3′ (SEQ ID NO: 11)) were ligated into SacI/ApaI-digested PX552 (Catalog number: 60958; Addgene), and the construct was designated as PX552Cj. The annealed and phosphorylated sgRNA oligonucleotides targeting the −124 C>T mutation in the TERT promoter locus (5′-GGCCCGGAAGGGGCTGGGCC-3′ (SEQ ID NO: 12)) and the protospacer-adjacent motif (PAM) (5′-GGAAACC-3′ spanning −136 to −142 bp in the TERT promoter region) were ligated into SapI-digested PX552Cj. CAG sgRNA (5′-GTTCCGCGTTACATAACTTA-3′ (SEQ ID NO: 13)) not targeting any loci of the human genome was used as a control. AAVs were produced via cotransfection of the pRC2-mi342 and pHelper plasmids into AAVpro 293T cells (Clontech Laboratories, Mountain View, Calif.).

U87 cells were infected with AAVs expressing CjABE or dCjCas9 under the guidance of sgRNAs with or without targeting of the TERT promoter mutation at MOI of 100. The cells were harvested at day 3 and day 10 after infection. Quantitative polymerase chain reaction (PCR) analyses were performed with actin mRNA as a normalization control.

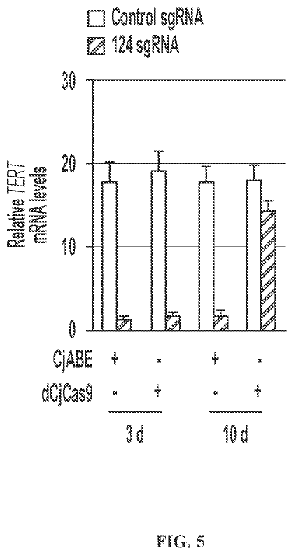

As shown in FIG. 5, although CjABE (sg RNA-guided and deactivated Campylobacter jejuni Cas9-fused adenine base editor (dCjCas9-ABE)) and dCjCas9 had comparable effect on inhibition of TERT mRNA expression at the day 3 after infection, CjABE has much improved and sustained long-term suppression of TERT expression than dCjCas9.

The above results demonstrate that CRISPR, CRISPR interference (CRISPRi), and programmable base editing (PBE)-based gene expression editing technology can be used to block the transcription of mutated TERT promoter in glioma and melanoma cells, leading to tumor cell senescence and proliferation arrest. Treatment of mice having glioma or melanoma with viruses expressing TERT promoter mutant-targeted CRISPR-Cas9-PBE inhibited tumor growth.

### Example 2—PBE of Mutated TERT Promoter Abrogates the Binding of ETS1 and GABPA to the Promoter

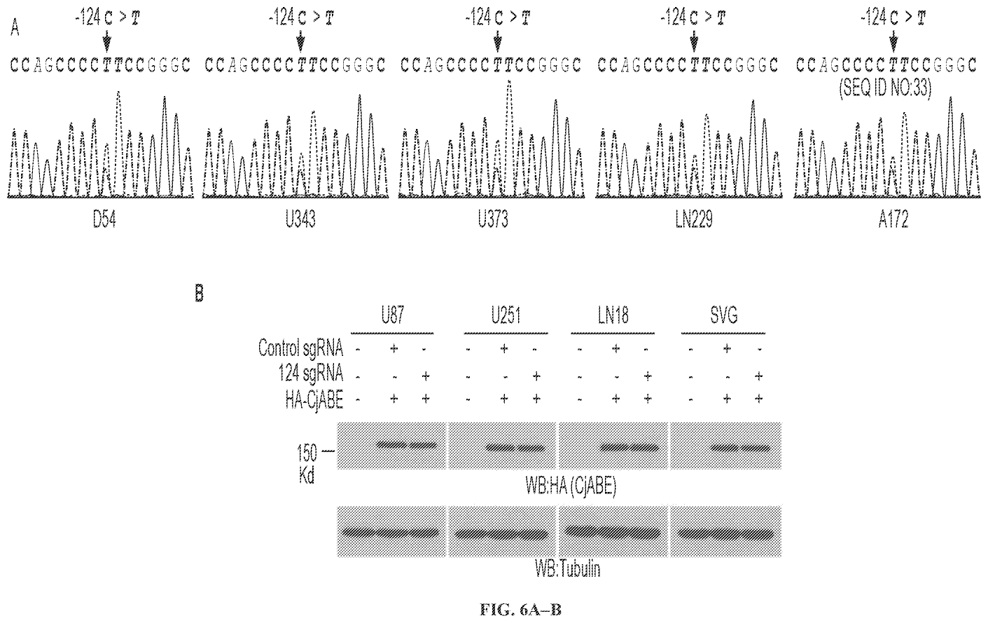

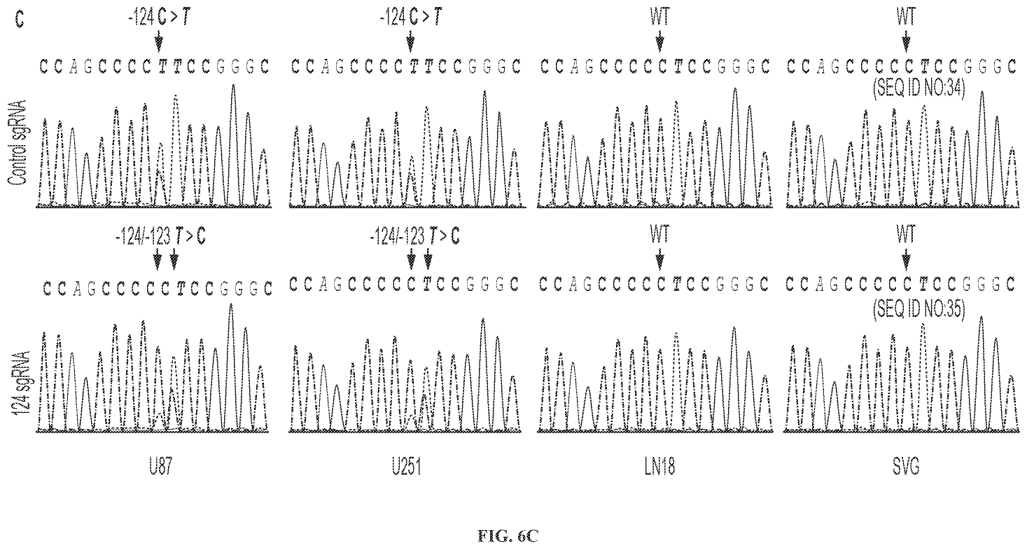

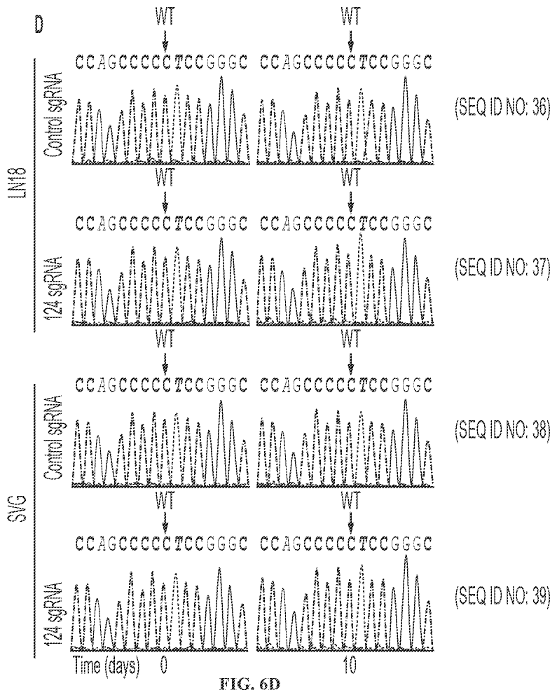

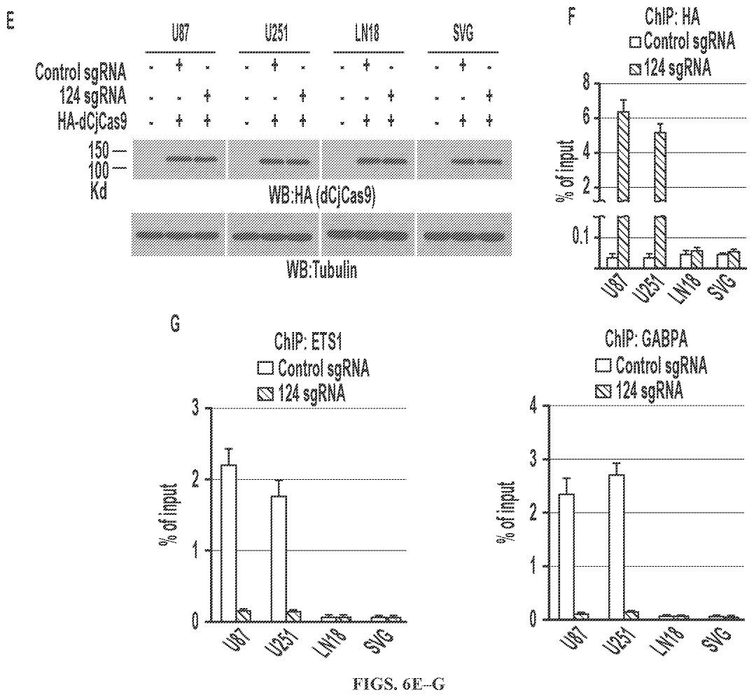

As telomerase reactivation is critical to tumor progression, the inventors explored whether somatic correction of the TERT promoter mutation would impact tumor maintenance, thus providing a strategy for cancer treatment. Sequencing was performed to identify GBM cell lines and SV40-immortalized human normal fetal glial (SVG) cells harboring the −124 C>T mutation (U87, U251, D54, U343, U373, LN229, and A172 GBM cells) or wildtype sequences (LN18 GBM and SVG cells) in the TERTpromoter (FIG. 1A; FIG. 6A). These results were in line with those of previous studies indicating that −124 C>T is a primary mutation in TERT promoter regions in GBM cells (Horn et al., 2013; Huang et al., 2013; Killela et al., 2013). Next, an sgRNA was designed with complementary sequences covering −124 C>T, which is nine nucleotides away from the protospacer-adjacent motif (PAM) 5′-GGAAACC-3′ spanning −136 to −142 bp in the TERT promoter region (Yamada et al., 2017). An adeno-associated virus (AAV) type 2 vector (Swiech et al., 2015) was constructed to express a hemagglutinin (HA)-tagged deactivated Cj Cas9-fused ABE protein (CjABE) containing a nuclear localization sequence, wild-type (WT) Escherichia coli tRNA-specific adenosine deaminase (ecTadA), evolved ecTadA (version 7.10), and dCjCas9 protein (FIG. 1B) (Gaudelli et al., 2017). This vector also expressed the sgRNA targeting the TERT-124 C>T mutation or a nontargeting sgRNA.

U87, U251, LN18, and SVG cells were infected with AAVs expressing HA-CjABE and specific and nonspecific sgRNA for 3 days and audited for HA-CjABE expression using immunoblot analysis (FIG. 6B). Gene-sequencing analyses demonstrated that expression of TERT −124 C>T sgRNA-guided CjABE but not nontargeting CjABE converted about 70% of −124 C>T mutations to −124 C in the mutated TERT promoter regions in U87 and U251 cells but not in LN18 or SVG cells with the WT TERT promoter (FIG. 6C). Of note, we also detected conversion of −123 T (adjacent to the mutated −124 nucleotide) to −123 C only in U87 and U251 cells. −123 T is eight nucleotides away from the PAM and within the correction range of CjABE. Time-course experiments demonstrated that expression of TERT −124 C>T sgRNA-guided CjABE in U87 and U251 cells converted almost 100% of −124 C>T mutations to −124 C (with no detectable uncorrected mutations) 10 days after the first-time AAV infection on days 0. Of note, 50% the adjacent −123 T was converted to 123 C (FIG. 1C) because the specific sgRNA bound to and affected only the mutated, not the WT, allele promoter. In contrast, the WT TERT promoter in LN18 and SVG cells was not affected in the same experimental setting (FIG. 6D). These results indicate that the designed PBE converts −123/124 T>C only in the mutated TERT promoter, not in the WT counterpart.

To determine whether the designed PBE affects the binding of ETS1 and GABPA to the mutated TERT promoter, chromatin immunoprecipitation (ChIP) assays were performed with anti-HA (FIG. 1D), anti-ETS1, and anti-GABPA (FIG. 1E) antibodies. CjABE was found to bind to the mutated TERT promoter regions in U87 and U251 cells but not the WT TERT promoter in LN18 or SVG cells in the presence of the sgRNA targeting −124 C>T but not of the nontargeting sgRNA (FIG. 1D). As expected, in the presence of nontargeting sgRNA expression, ETS1 and GABPA bound to the TERT promoter regions in U87 and U251 cells but not the TERT promoter region in LN18 or SVG cells (FIG. 1E). Notably, this binding of ETS1 and GABPA in the mutated TERT promoter regions was abrogated by expression of the TERT −124 C>T sgRNAs and CjABE (FIG. 1E), indicating that −123/124 T>C abrogates the binding of ETS1 and GABPA to the TERT promoter. Notably, expression of TERT −124 C>T sgRNA-guided dCjCas9 alone (FIG. 6E), without ecTadA expression, demonstrated binding of dCjCas9 to the mutated TERT promoter region (FIG. 6F) and abrogation of the binding of ETS1 and GABPA to this region (FIG. 6G). These results suggested that binding of dCjCas9 to the mutated TERT promoter physically blocks the binding of ETS1 and GABPA to this region and that expression of fused CjABE gains an additional function: permanent correction of mutated TERT −124 C>T. Together, these results indicated that sgRNA-guided binding of CjABE to the mutated TERT promoter regions corrects TERT −124 C>T mutation and prevents binding of ETS1 and GABPA to the TERT promoter in GBM cells.

### Example 3—PBE of the Mutated TERT Promoter Inhibits TERT Expression

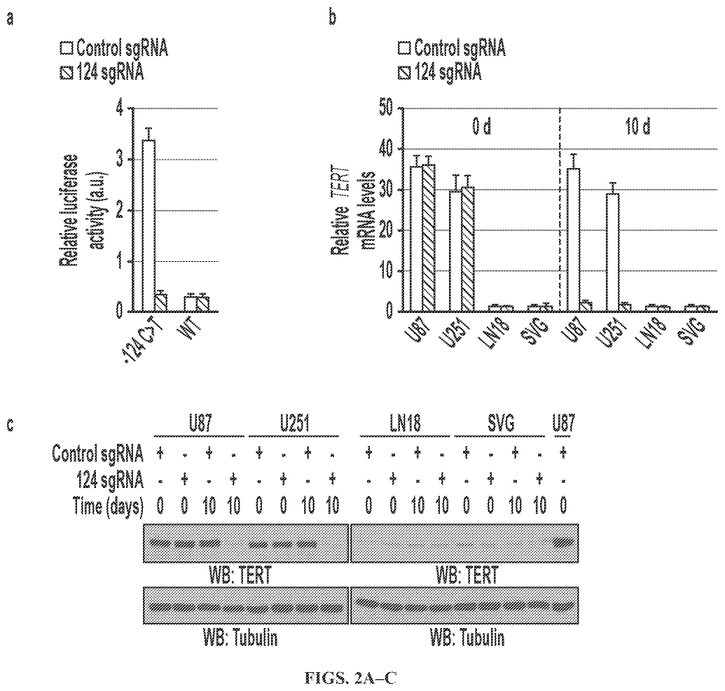

To determine the effect of TERT promoter-specific PBE on the activity of the mutated TERT promoter, a vector was constructed to express luciferase driven by the WT or mutated TERT promoter. In line with previous reports (Horn et al., 2013; Huang et al., 2013), the mutated TERT promoter had substantially greater activity than its WT counterpart did. However, this enhanced activity was abolished by expression of the TERT −124 C>T sgRNA-guided CjABE (FIG. 2A). In addition, elevated TERT mRNA (FIG. 2B) and protein (FIG. 2C) expression in U87 and U251 cells but not LN18 or SVG cells was downregulated by TERT −124 C>T sgRNA-guided CjABE expression. Thus, sgRNA-guided conversion of −123/124 T>C by CjABE at the mutated TERT promoter regions inhibits TERT transcription and protein expression.

### Example 4—Mutated TERT Promoter-Targeted PBE Reduces Telomere Lengths and Induces Tumor-Cell Senescence and Proliferation Inhibition

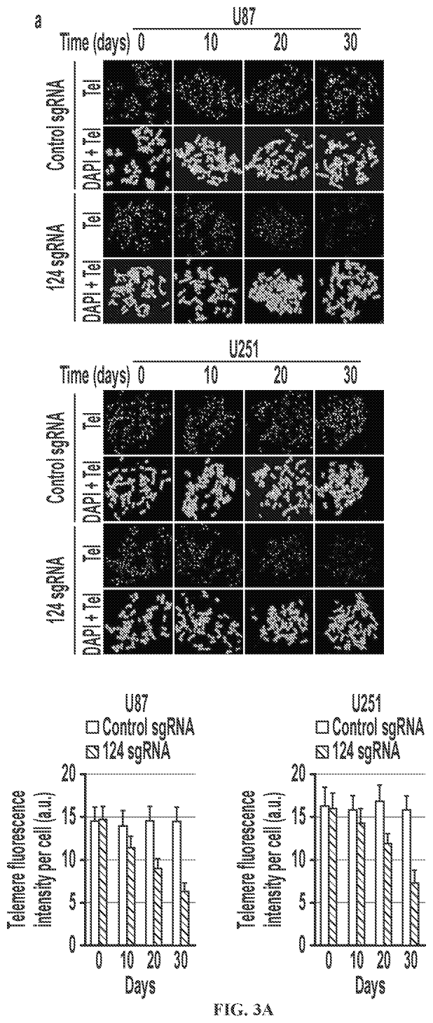

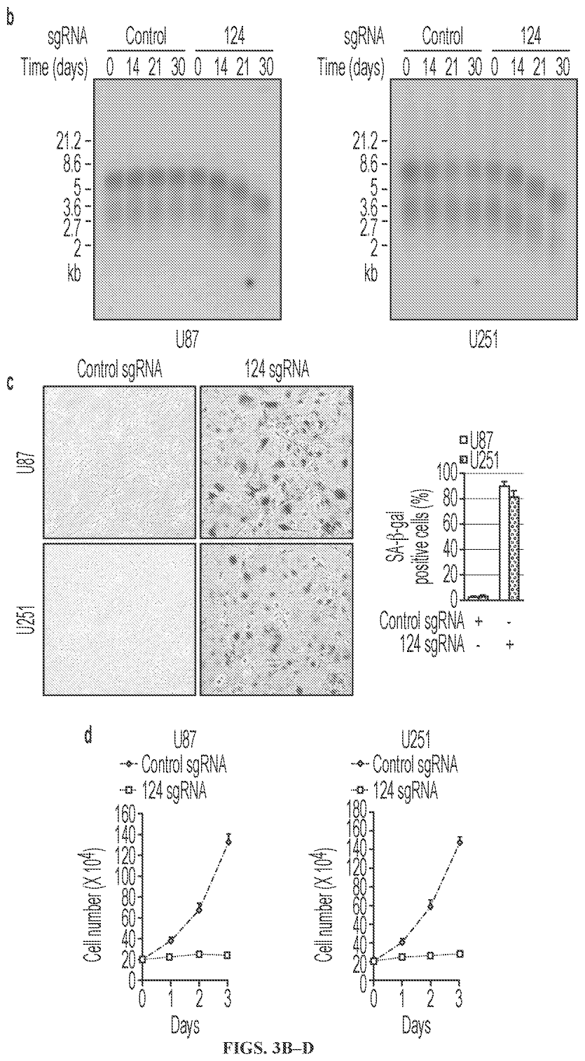

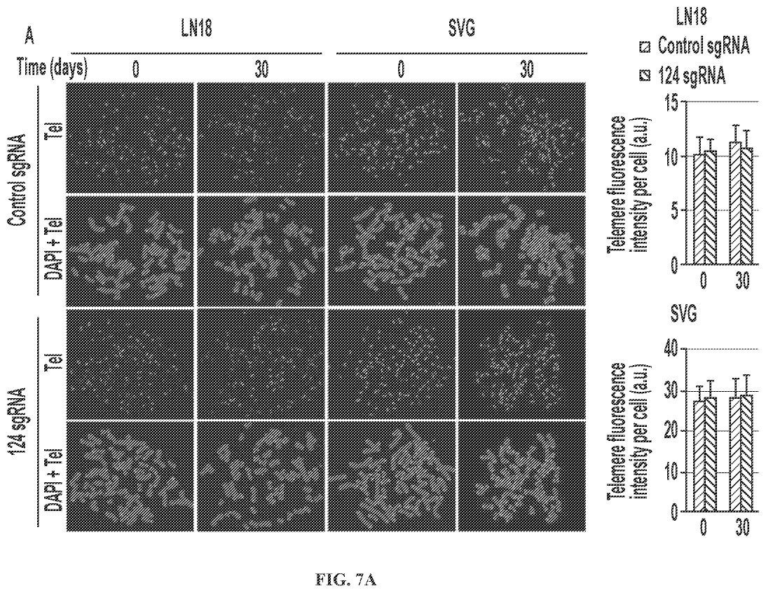

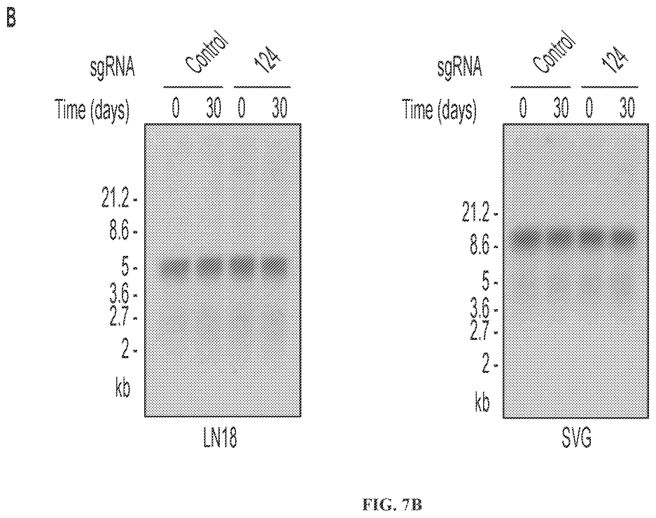

Next, telomere lengths were determined in GBM cells using quantitative fluorescence in situ hybridization (QFISH). Telomere lengths decreased more rapidly in U87 and U251 cells expressing TERT −124 C>T sgRNA-guided CjABE than in cells expressing nontargeting CjABE (FIG. 3A). In contrast, telomere lengths in LN18 and SVG cells were not affected by TERT −124 C>T sgRNA-guided CjABE expression (FIG. 7A). Similar results were obtained using telomere restriction fragment (TRF) analyses, which show telomere lengths by Southern blotting (FIG. 3B; FIG. 7B). Correspondingly, senescence-associated biomarker β-galactosidase-activity staining was evident in U87 and U251 cells with TERT −124 C>T sgRNA-guided CjABE expression, but not in those cells with nontargeting CjABE expression (FIG. 3C). In addition, TERT −124 C>T sgRNA-guided CjABE expression largely inhibited the proliferation of U87 and U251 cells (FIG. 3D). Thus, consistent with the role of telomerase in cell immortality, mutant TERT promoter-specific conversion of −123/124 T>C by CjABE provokes glioma-cell senescence and proliferative arrest.

### Example 5—Mutated TERT Promoter-Targeted PBE Inhibits Brain Tumorigenesis In Vivo

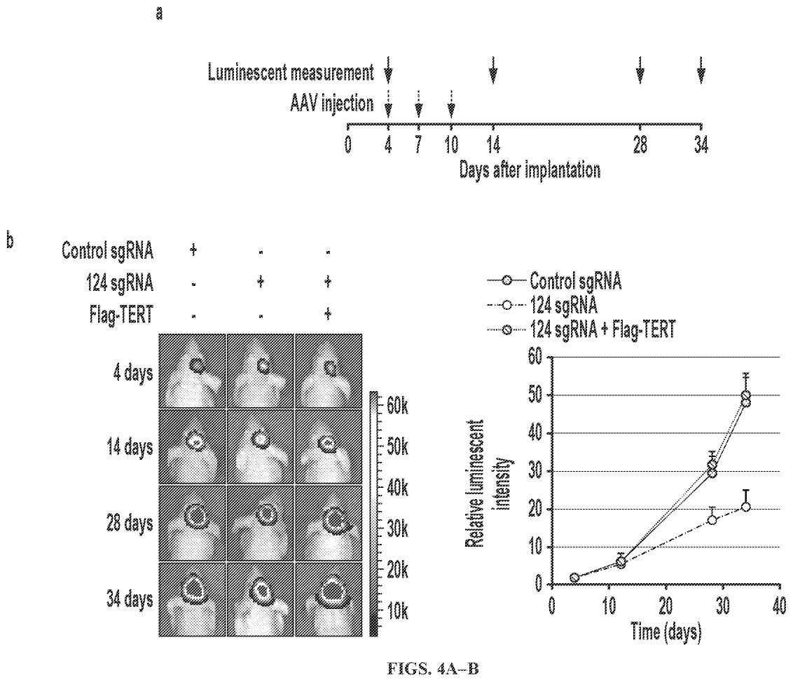

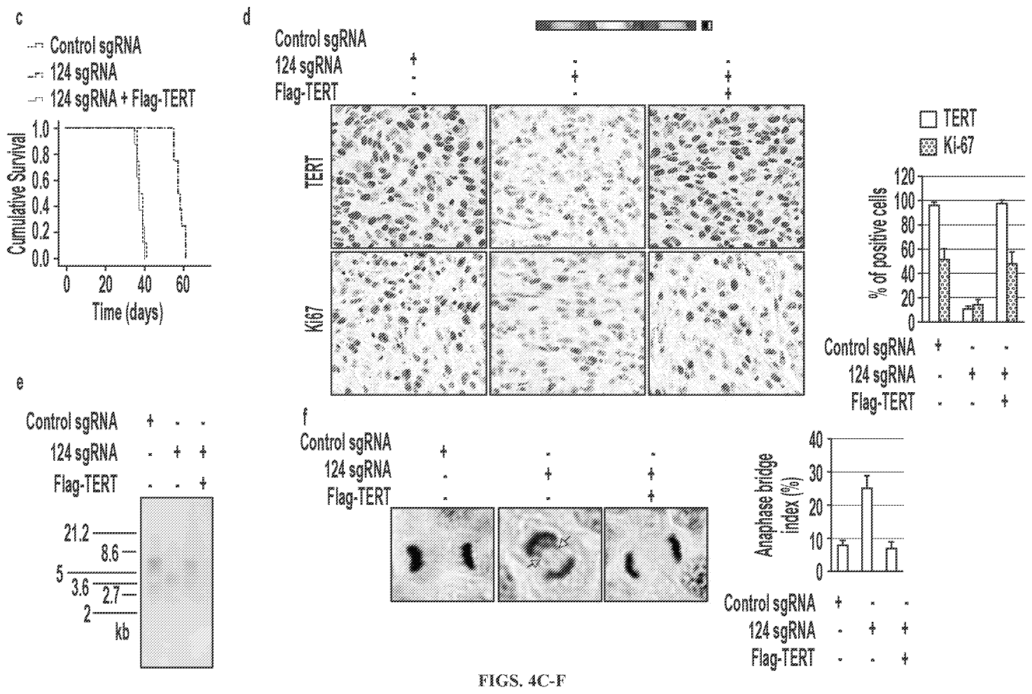

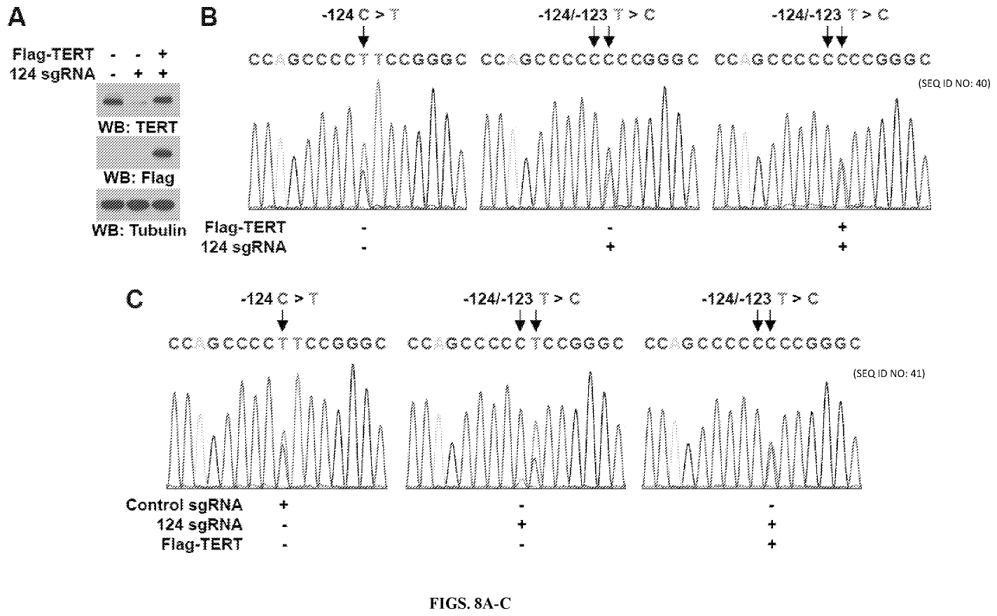

To determine the therapeutic potential of TERT promoter mutation-targeted PBE, U87 cells expressing luciferase were injected intracranially into athymic nude mice, which were then subjected to three times injections of AAVs expressing TERT −124 C>T sgRNA-guided or nontargeting CjABE (FIG. 4A). In addition, another group of athymic nude mice were injected with U87 cells containing a PBE-corrected mutated TERT promoter and having reconstituted expression of Flag-TERT (FIGS. 8A-B), and these mice were received the same AAV injection. Gene-sequencing analyses of tumor samples demonstrated that injection of the AAVs expressing the TERT −124 C>T sgRNA-guided CjABE but not nontargeting CjABE successfully corrected the −124 T>C at the mutated TERT promoter regions in tumors derived from U87 cells (FIG. 8C) without affecting the TERT promoter sequence in tumors derived from U87 cells with reconstituted expression of TERT (FIG. 8C). Bioluminescent imaging demonstrated that injection of AAVs expressing TERT −124 C>T sgRNA-guided CjABE resulted in considerable reduction in glioma growth (FIG. 4B), which was accompanied by considerably prolonged survival time (FIG. 4C). Of note, reconstituted expression of Flag-TERT in U87 cells with a PBE-corrected mutated TERT promoter restored tumor growth and decreased survival time to levels comparable with those for U87 cells with expression of nontargeting CjABE (FIGS. 4B and C). These results are consistent with the view that the effect of TERT promoter mutation-targeted PBE on tumor growth is not caused by off-target DNA edits. Thus, correction of the mutated TERT promoter in glioma cells inhibits tumor growth and prolongs overall survival.

Immunohistochemical analyses with anti-TERT and anti-Ki67 antibodies and TRF analyses revealed that tumor samples containing a PBE-modified TERT promoter had decreased TERT and Ki67 expression (FIG. 4D) as well as reduced telomere length (FIG. 4E). Notably, these effects were reversed by reconstituted expression of TERT.

In addition, hematoxylin and eosin staining of tumor samples was performed to assess the formation of anaphase bridges, a hallmark of telomere dysfunction, which results from uncapped chromosomes with short dysfunctional telomeres, leading to unstable chromosome rearrangements prone to bridging at anaphase (Tusell et al., 2010). A much higher rate of formation of anaphase bridges was observed in tumors infected with TERT −124 C>T sgRNA-guided CjABE-expressing AAVs than in those infected with control AAVs (FIG. 4F). The occurrence of anaphase bridges in the tumors infected with TERT −124 C>T sgRNA-guided CjABE-expressing AAVs was reduced upon reconstitution of TERT expression. Thus, mutant TERT promoter-specific conversion of −123/124 T>C by CjABE disrupts telomere function and inhibits gliomagenesis.

Immortal cell growth is a hallmark of cancer, and may be enabled by telomerase-mediated telomere maintenance catalyzed by TERT (Shay 2016; Arndt and MacKenzie 2016). Telomerase is frequently activated in cancer cells (Shay 2016; Marian et al., 2010). The high frequency of somatic mutations of the TERT promoter in primary GBMs (83%) (Horn et al., 2013; Huang et al., 2013; Nault et al., 2013) indicates that the approaches disclosed herein may be used to quell telomerase activity, e.g., in glioma maintenance. To date, TERT-targeted therapies have seen limited development (Shay 2016). Here, CRISPR, CRISPRi, and PBE (Doudna et al., 2014; Gaudelli et al., 2017; Hsu et al., 2014) approaches were used to convert the mutated TERT promoter −124C>T to −124C via CjABE. This somatic modification blocked association of ETS family members with the TERT promoter, reduced TERT transcription and protein expression, and provoked GBM-cell senescence and proliferation inhibition. The PBE approach efficiently reduced TERT transcription and inhibited tumor growth. The translational application of the PBE approach is enhanced by avoidance of potential mutations created by CRISPR-induced DNA repair (Gaudelli et al., 2017).

PBE can result in fewer off-target genome modifications than a current Cas9 nuclease-based method (Gaudelli et al., 2017). Although it was observed that −124C-adjacent −123T was converted to −123C in tumor cells, this conversion occurred only within the mutated TERT promoter and retained the inhibitory effect on ETS binding to the TERT promoter. Expression of CjABE had no effect on GBM or SVG cells with the WT TERT promoter but specifically blocked TERT expression in GBM cells with TERT promoter mutations. In addition, this inhibition was abrogated by reconstitution of Flag-TERT expression, demonstrating the specificity of the PBE approach used. Local injection of AAVs expressing sgRNA-guided CjABE inhibited the growth of brain tumors with TERT promoter mutations, demonstrating higher therapeutic specificity for TERT promoter-mutated tumors than that of other approaches, such as use of small molecular compounds, short hairpin RNA, and small interfering RNA (without wishing to be bound by any theory, it is anticipated that these other approaches may affect highly proliferative normal cells and create unwanted side effects). Considering the very limited success in treating human GBM, which is reflected by a median survival duration of about 14 months (Cloughesy et al., 2014; Yuan et al., 2016), these findings that targeting the critical tumor maintenance role of TERT promoter mutation-mediated telomerase activation may be used to treat GBM, and this approach contrasts with the meager preclinical and clinical impact of targeting many other driver mutations, including activating epidermal growth factor receptor mutations, in GBM (Wykosky et al., 2011). These results indicate that PBE can be used for targeting tumor maintenance mutations in cancer patients.

### Example 6—Materials and Methods

Materials: Rabbit polyclonal antibodies recognizing telomerase (ab32020, 1:1000) were obtained from Abcam (Cambridge, UK). Rabbit polyclonal antibodies recognizing Ki67 (AB9260, 1:1000) and GABPA (ABE1845, 1:100) were obtained from EMD Millipore (Burlington, Mass.). Mouse monoclonal antibodies against tubulin (sc-5286, clone B-7; 1:2000) and ETS1 (sc-111, 1:100) were purchased from Santa Cruz Biotechnology (Santa Cruz, Calif.). A mouse monoclonal antibody against Flag (F3165, clone M2; 1:5000) was purchased from Sigma (St. Louis, Mo.). Horseradish peroxidase-conjugated goat anti-mouse and anti-rabbit secondary antibodies were obtained from Thermo Fisher Scientific (Waltham, Mass.). HyFect transfection reagents were obtained from Denville Scientific (Holliston, Mass.).

Cell Lines and Cell Culture Conditions: The human GBM cell lines U87, U251, LN18, D54, U343, U373, LN229, and A172; the human fetal glial cell line SVG; and luciferase-expressing U87 cells (a gift from Dr. Chun Li, MD Anderson) were maintained in Dulbecco's modified Eagle's medium supplemented with 10% bovine calf serum (HyClone).

Transfection: Cells were seeded into a 60-mm dish at a density of 4×10⁵ 18 hr prior to transfection. Transfection was performed using HyFect reagents according to the manufacturer's instructions. Transfected cultures were selected with hygromycin (200 μg/ml) for 14 days, and antibiotic-resistant colonies were selected, pooled, and expanded for further analyses under selective conditions.

Immunoblot Analysis: Proteins were extracted from cultured cells, and immunoblot analyses of the proteins with corresponding antibodies were performed as described previously (1). The band intensity was quantified using the Image Lab software program (Bio-Rad Laboratories, Hercules, Calif.).

Genotyping of the TERT Promoter: Genomic DNA was extracted from cell lines using a DNeasy Kit (QIAGEN, Hilden, Germany). DNA fragments spanning the mutation region of the human TERT promoter were amplified using PCR with a pair of primers (forward, CACATCATGGCCCCTCCCTC (SEQ ID NO: 14); reverse, GAAGCCGAAGGCCAGCACG (SEQ ID NO: 15)). The sequences of the PCR-amplified TERT promoter region were determined via Sanger sequencing.

AAV Packaging: A plasmid expressing dCjCas9 was generated by introducing mutations of D8A and H559A into WT CjCas9 in a PX404 plasmid (Catalog number: 68338; Addgene, Cambridge, Mass.) using a QuikChange site-directed mutagenesis kit (Stratagene, La Jolla, Calif.) and was designated as PX404d (2). The DNA sequence encoding HA-tagged WT ecTadA, TadA (7.10), and the N-terminus (1-166 amino acids) of dCjCas9 were synthesized by Thermo Fisher Scientific. An AgeI/PflMI-digested fragment containing the HA-ecTadA (WT)-ecTadA (7.10)-dCjCas9 (1-166 amino acids) cassette was ligated into AgeI 1PflMI-digested PX404d, and the construct was designated as PX404TadA. The open reading frame of HA-ecTadA (WT)-ecTadA (7.10)-dCjCas9 was amplified from PX404TadA using high-fidelity PCR and then ligated into the AgeI/NotI-digested adeno-associated viral vector pAAV-EFS-SpCas9 (Catalog number: 200932; Addgene), and the construct was designated as pAAV-CjABE. To construct the adeno-associated viral vector expressing sgRNA for CjCas9, annealed and phosphorylated sgRNA backbone oligonucleotides (forward, 5′-CTTCTGTTTTAGTCCCTGAAGGGACTAAAATAAAGAGTTTGCGGGACTCTGCGGG GTTACAATCCCCTAAAACCGCTTTTTTTCTAGACTGCAGAGGGCC-3′ (SEQ ID NO: 10); reverse, 5′-CTCTGCAGTCTAGAAAAAAAGCGGTTTTAGGGGATTGTAACCCCGCAGAGTCCC GCAAACTCTTTATTTTAGTCCCTTCAGGGACTAAAACAGAAGAGCT-3′ (SEQ ID NO: 11)) were ligated into SacI/ApaI-digested PX552 (Catalog number: 60958; Addgene), and the construct was designated as PX552Cj. The annealed and phosphorylated sgRNA oligonucleotides targeting the −124 C>T mutation in the TERT promoter locus (5′-GGCCCGGAAGGGGCTGGGCC-3′ (SEQ ID NO: 12)) and the protospacer-adjacent motif (PAM) (5′-GGAAACC-3′ spanning −136 to −142 bp in the TERT promoter region) were ligated into SapI-digested PX552Cj. CAG sgRNA (5′-GTTCCGCGTTACATAACTTA-3′ (SEQ ID NO: 13)) not targeting any loci of the human genome was used as a control. AAVs were produced via cotransfection of the pRC2-mi342 and pHelper plasmids into AAVpro 293T cells (Clontech Laboratories, Mountain View, Calif.). Infectious AAVs were isolated from AAV-producing 293T cells 3 days after transfection and purified using an AAVpro Purification Kit (Clontech Laboratories). Titration of AAVs was determined using an AAVpro Titration Kit (Clontech Laboratories) according to the manufacturer's instructions.

Reverse transcriptase-PCR: Reverse transcriptase-PCR analyses were performed as described previously (3). Briefly, total RNA was extracted from cultured tumor cells using TRIzol reagent (Invitrogen, Carlsbad, Calif.) according to the manufacturer's instructions. Total RNA (1 μg) was used for cDNA synthesis in a 20-μ1 reaction with an iScript cDNA synthesis kit (Bio-Rad Laboratories). One microliter of the cDNA library was used in a 25-μ1 PCR. Fast SYBR Green Master Mix (Bio-Rad Laboratories) was used to determine the threshold cycle value for each sample using a CFX96 real-time PCR detection system (Bio-Rad Laboratories). β-actin served as the normalization gene in these studies. The relative expression levels for the target genes were determined using the 2^{ΔCt} method (the threshold cycle for β-actin minus the threshold cycle for the target gene). The sequences of the PCR primers used for amplification of β-actin and TERT were as follows: β-actin-F, GAGATCACTGCCCTGGCACC (SEQ ID NO: 16); β-actin-R, GATGGAGGGGCCGGACTCG (SEQ ID NO: 17); TERT-F, CAAGTTCCTGCACTGGCTGATG (SEQ ID NO: 18); and TERT-R, CAAGTGCTGTCTGATTCCAATGC (SEQ ID NO: 19).

DNA Constructs and Mutagenesis: The pGL3-TERT plasmid was constructed via insertion of a PCR-amplified human TERT promoter into a pGL3-Basic luciferase reporter vector via digestion with the KpnI and HindIII restriction enzymes. The pGL3-TERT plasmid containing −124 C>T was constructed using a QuikChange site-directed mutagenesis kit (Stratagene). pcDNA3.1 Flag-TERT plasmid was constructed via insertion of a PCR-amplified human TERT cDNA into an NheI/NotI-digested pcDNA 3.1/hygro (+) vector.

ChIP Assay: ChIP assay analyses were performed as described previously (4) using a SimpleChIP Enzymatic Chromatin IP Kit (Cell Signaling Technology, Danvers, Mass.). Chromatin prepared from cells in a 10-cm dish was used to determine the total DNA input and was incubated overnight with specific antibodies or normal mouse IgG. The PCR primer sequences were as follows: forward, CCTTCCAGCTCCGCCTCCTC (SEQ ID NO: 20); reverse, CGGGGCCGCGGAAAGGAAG (SEQ ID NO: 21).

Luciferase Assay: To determine the effect of mutation of the TERT promoter on luciferase gene transcription, 5×10⁵ U87 cells seeded in 60-mm dishes were transfected with pGL3-Basic luciferase reporter plasmids containing a WT or mutated TERT promoter. The cells were infected with AAVs (MOI=100) 16 hr after transfection. Forty-eight hours after virus infection, luciferase assays were performed using a Dual Luciferase Reporter Assay System (Promega, Madison, Wis.) and normalized for transfection efficiency via cotransfection of Renilla luciferase.

QFISH Analysis of Telomere Length: Cells were infected with AAVs (MOI=100) expressing CjABE and sgRNA with or without targeting of the mutated TERT promoter for the indicated times. QFISH analyses of telomere length were then performed as described previously (5). Briefly, cells were arrested in metaphase via treatment with 1 μg/ml colcemid for 90 min. Trypsinized cells were incubated in ice-cold 0.56% KCl solution, fixed with methanol:acetic acid (3:1), and spread on glass slides. The slides were left to air-dry overnight. The next day, the slides were rehydrated with 2× saline sodium citrate (SSC) buffer and treated with 100 μg/ml RNase A for 1 hr, and pepsin (50 U/ml) was diluted in 10 mM HCl for 10 min at 37° C. After fixation in 4% formaldehyde for 5 min, the slides were dehydrated in 70%, 85%, and 100% (v/v) ethanol for 1 min each and then air-dried. Metaphase chromosome spreads were denatured via heating at 85° C. for 5 min before hybridization with a 200-nM Tel C-Cy3 PNA probe (cat. #F1002; PNA Bio Inc., Newbury Park, Calif.) diluted in 70% formamide/10 mM Tris-HCl (pH 7.4) for 2 hr at 37° C. Following hybridization, the slides were washed twice for 15 min each in 70% formamide/10 mM Tris-HCl (pH 7.4) followed by washing three times with 2×SSC buffer for 5 min each. The chromosomes were counterstained with 1 μg/ml 4′,6-diamidino-2-phenylindole and mounted using ProLong Gold antifade reagent (Thermo Fisher Scientific). Images were acquired using an FLV1000 inverted microscope equipped with a 63× oil objective (Olympus Scientific Solutions, Waltham, Mass.). Afterward, images were imported into the ImageJ and Photoshop CS5 (Adobe Systems, San Jose, Calif.) software programs for manual quantitation.

TRF Analysis: TRF analyses were performed as described previously (6) with some modifications. Briefly, genomic DNA was isolated from indicated cells and tissues using a QIAamp DNA Mini Kit (Catalog number: 51304; QIAGEN) according to the manufacturer's instructions. The isolated genomic DNA (2 μs) was digested with HinfI and RsaI (20 U each) overnight at 37° C. The resulting DNA was normalized and separated via electrophoresis with a 0.8% agarose gel. The gel was denatured in 0.5 M NaOH and 1.5 M NaCl for 30 min with shaking at 25° C., neutralized by washing twice with 1 M Tris (pH 7.5) and 3 M NaCl for 15 min with shaking at 25° C., and transferred to a nylon membrane for Southern blotting. The membrane was prehybridized in Church buffer (1% bovine serum albumin, 1 mM EDTA, 0.5 M NaPO₄ pH 7.2, 7% sodium dodecyl sulfate) for 30 min and then hybridized with a ³²P-end-labeled (TTAGGG) telomeric probe for 2 hr at 42° C. followed by washing three times with 2×SSC buffer for 30 min each at 42° C. and one time with 2×SSC buffer and 1% sodium dodecyl sulfate for 30 min at 25° C. before autoradiography.

DNA Probe: The polyacrylamide gel electrophoresis-purified telomeric probe (TTAGGG) was radioactively labeled with ³²Pusing T4 polynucleotide kinase (Catalog number: M0201; New England BioLabs, Ipswich, Mass.). Briefly, 50 pmol of the telomeric probe mixed with 50 pmol of [γ-³²P] ATP (cat. #BLU002H250UC; PerkinElmer, Waltham, Mass.) and 20 U of T4 polynucleotide kinase in a total volume of 20 μl of kinase buffer (25 mM Tris-HCl, pH 7.5, 5 mM β-glycerophosphate, 2 mM DTT, 0.1 mM Na₃VO₄, 10 mM MgCl₂) was incubated for 30 min at 37° C. The reaction was terminated via heating for 20 min at 65° C.

Cellular Senescence Staining: Cells were infected with AAVs (MOI=100) expressing CjABE and sgRNA with or without targeting of the mutated TERT promoter for the indicated periods. Infected cells (2×10⁵) suspended in 2 ml of medium were then seeded in six-well plates, maintained in Dulbecco's modified Eagle's medium with 10% bovine calf serum for 24 hr, and stained for senescence using a β-Galactosidase Staining Kit (Cell Signaling Technology). The stained cells were mounted with 70% glycerol, and the percentage of β-galactosidase-positive cells was calculated.

Cell Proliferation Assay: Cells were infected with AAVs (MOI=100) expressing CjABE and sgRNA with or without targeting of the mutated TERT promoter at the indicated time points. Infected cells (2×10⁵) suspended in 2 ml of medium were then seeded in six-well plates and maintained in Dulbecco's modified Eagle's medium with 10% bovine calf serum. The cells in each well were trypsinized and counted at the indicated times after seeding.

Tumor Xenografts: An implantable guide-screw system that allows for precise multiple intratumoral administration of therapeutic agents was used in our orthotopic brain tumor experiments, as described previously (7). GBM cells (2×10⁵) in 5 μl of Dulbecco's modified Eagle's medium were injected intracranially into female 4-week-old athymic nude mice (8 mice/group). AAV-based treatment was initiated 4 days after tumor-cell injection. Specifically, AAVs (1×10¹⁰ viral particles in 10 μl of phosphate-buffered saline) were delivered via intracranial administration at the indicated times. Survival of each mouse was assessed by examining clear signs of morbidity after injection of tumor cells. The animals used in this study were administered in accordance with relevant institutional and national guidelines and regulations.

Bioluminescent Imaging: Bioluminescent imaging of mice was performed using an IVIS Lumina System coupled with the Living Image data-acquisition software program (Xenogen Corporation, Alameda, Calif.) (8). Briefly, D-luciferin (450 mg/kg; Cayman Chemical, Ann Arbor, Mich.) in 250 μl of phosphate-buffered saline was subcutaneously injected into the neck region in mice. Images of the mice were acquired 10-20 min after D-luciferin administration, and peak luminescent signals were recorded. The tumor-emanating bioluminescent signal was quantified by measuring photon flux within a region of interest using the Living Image software program.

Histologic Evaluation and Immunohistochemical Staining: Mouse tumor samples were fixed, paraffin-embedded, sectioned (5 μm), and stained with Mayer's hematoxylin and eosin (BioGenex, Fremont, Calif.) (9). Slides were then mounted using Universal mount (Research Genetics, Huntsville, Ala.) and examined under a light microscope.

Sections of paraffin-embedded xenograft tissue were stained with antibodies against TERT or Ki67 or with nonspecific IgG as a negative control. Immunohistochemical staining of the sections was performed using a VECTASTAIN ABC kit (Vector Laboratories, Burlingame, Calif.) according to the manufacturer's instructions.

Statistical Analysis and Reproducibility: The mean values obtained for the control and experimental groups were analyzed for significant differences. Pair-wise comparisons were performed using the two-tailed Student t-test. P values less than 0.05 were considered significant.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. 2019/0093099
U.S. 2013130248
U.S. 20150376587
U.S. 2015218573
U.S. 2015356239
U.S. 2015356239
U.S. 2019/0093099
U.S. 2019/0093099
U.S. App. No. 61/874,682
U.S. App. No. 61/874,746
U.S. Pat. No. 5,994,136
U.S. Pat. No. 5,994,136
U.S. Pat. No. 6,013,516
EP1507865
PCT/EP2000/011690
WO/2001/038547
WO2015089351
WO2015106004
WO2015157534
WO2015200555
Arndt and MacKenzie, New prospects for targeting telomerase beyond the telomere. Nature reviews. Cancer 16, 508-524, 2016.
Aubert et al., 2008.
Bell et al. 2015.
Biagioni et al. J Biol Eng. 2018; 12: 33, 2018.
Cesare et al., 2010.
Chiba et al., 2015.
Chylinski et al., “The tracrRNA and Cas9 families of type II CRISPR-Cas immunity systems,” RNA Biology 10:5, 726-737, 2013.
Cloughesy et al., 2014; Yuan et al., 2016
Cloughesy et al., Glioblastoma: from molecular pathology to targeted treatment. Annu Rev Pathol 9, 1-25, 2014.
Deltcheva et al., Nature 471:602-607, 2011.
Doudna et al., Genome editing. The new frontier of genome engineering with CRISPR-Cas9. Science 346, 1258096, 2014.
Ferretti et al., Proc. Natl. Acad. Sci. U.S.A. 98:4658-4663, 2001.
Gaudelli et al., Programmable base editing of A*T to G*C in genomic DNA without DNA cleavage. Nature 551, 464-471, 2017.
Gilbert et al. 2013.
Green and Sambrook, Molecular Cloning: A Laboratory Manual (4^{th} ed., Cold Spring Harbor Laboratory Press, Cold Spring Harbor, N.Y., 2012).
Horn et al., TERT promoter mutations in familial and sporadic melanoma. Science 339, 959-961, 2013.
Hsu et al., Development and applications of CRISPR-Cas9 for genome engineering. Cell 157, 1262-1278, 2014.
Huang et al., Highly recurrent TERT promoter mutations in human melanoma. Science 339, 957-959, 2013.
Jinek et al., Science 337:816-821, 2012.
Killela et al., TERT promoter mutations occur frequently in gliomas and a subset of tumors derived from cells with low rates of self-renewal. Proceedings of the National Academy of Sciences of the United States of America 110, 6021-6026, 2013.
Kinde et al., 2013.
Komor A C, et al. Improved base excision repair inhibition and bacteriophage Mu Gam protein yields C:G-to-T:A base editors with higher efficiency and product purity. Sci Adv.; 3:eaao4774. [PubMed: 28875174], 2017
Li et al., 2015.
Lin et al. Adv Sci (Weinh). 5(4): 1700611, April 2018.
Lino et al. Drug Deliv., 25(1):1234-1257, November 2018.
Maeder et al. 2013.
Marian et al., The telomerase antagonist, imetelstat, efficiently targets glioblastoma tumor-initiating cells leading to decreased proliferation and tumor growth. Clin Cancer Res 16, 154-163., 2010.
Nault et al., High frequency of telomerase reverse-transcriptase promoter somatic mutations in hepatocellular carcinoma and preneoplastic lesions. Nature communications 4, 2218, 2013.
Qi et al., “Repurposing CRISPR as an RNA-Guided Platform for Sequence-Specific Control of Gene Expression,” Cell. 28; 152(5):1173-83, 2013.
Remington: The Science and Practice of Pharmacy, 21^{st} Ed., Lippincott Williams and Wilkins, 2005.
Remington's Pharmaceutical Sciences, 18th Ed. Mack Printing Company, 1990, pp. 1289-1329, 1990.
Sambrook et al., 2001.
Shay, Role of Telomeres and Telomerase in Aging and Cancer. Cancer discovery 6, 584-593, 2016.
Stern et al. 2015.
Swiech et al., In vivo interrogation of gene function in the mammalian brain using CRISPR-Cas9. Nat Biotechnol 33, 102-106, 2015.
The tracrRNA and Cas9 families of type II CRISPR-Cas immunity systems,” RNA Biology 10:5, 726-737, 2013.
Tusell et al., Different outcomes of telomere-dependent anaphase bridges. Biochemical Society transactions 38, 1698-1703, 2010.
Wykosky et al., Therapeutic targeting of epidermal growth factor receptor in human cancer: successes and limitations. Chinese journal of cancer 30, 5-12, 2011.
Xi et al., 2015.
Yamada et al., Crystal Structure of the Minimal Cas9 from Campylobacter jejuni Reveals the Molecular Diversity in the CRISPR-Cas9 Systems. Molecular cell 65, 1109-1121 e1103, 2017.
Yuan et al., TERT mutation in glioma: Frequency, prognosis and risk. J Clin Neurosci 26, 57-62, 2016.

What is claimed is:

1. A method of treating a cancer in a mammalian subject comprising administering to the subject a CRIPSR therapy to reverse a point mutation in a telomerase reverse transcriptase (TERT) promoter in the cancer.

2. The method of claim 1, wherein the point mutation is a C>T point mutation.

3. The method of claim 2, wherein the point mutation is −124C>T, −228C>T, or −250C>T.

4. The method of any one of claims 1-3, wherein the CRISPR therapy comprises administering a nucleic acid encoding a sgRNA-guided Cas9 or nuclease-deactivated Cas9 (dCas9) to the subject.

5. The method of claim 4, wherein the nucleic acid is delivered via a viral vector.

6. The method of claim 5, wherein the viral vector is an adenovirus, adeno-associated virus, retrovirus, lentivirus, Newcastle disease virus (NDV), or lymphocytic choriomeningitis virus (LCMV).

7. The method of claim 4, wherein the nucleic acid is delivered via an exosome, lipid-based transfection, nanoparticle, or cell-based delivery system.

8. The method of claim any one of claims 1-3, wherein the CRISPR therapy comprises administering a sgRNA-guided deactivated Cas9 that is fused to an adenine base editor (ABE) to the subject.

9. The method of claim 8, wherein the deactivated Cas9 is a deactivated Campylobacter jejuni Cas9, S. pyogenes Cas9, or S. thermophiles Cas9.

10. The method of claim 9, wherein the deactivated Cas9 is a deactivated Campylobacter jejuni Cas9.

11. The method of any one of claims 8-10, wherein the sgRNA-guided deactivated Cas9 that is fused to an adenine base editor (ABE) is further fused to a cell penetrating peptide (CPP) or nuclear localization signal.

12. The method of any one of claims 8-10, wherein the sgRNA-guided deactivated Cas9 that is fused to an adenine base editor (ABE) is delivered via a viral vector.

13. The method of claim 12, wherein the adenine base editor comprises a mutation at one or more amino acid positions corresponding to amino acids that are involved in H-bond contacts with tRNA in a wild-type adenosine deaminase, preferably wherein the wild-type adenosine deaminase is a TadA deaminase.

14. The method of claim 13, wherein the TadA deaminase comprises the mutations (A106V and D108N), or three or more of: W23R, H36L, (P48S or P48A), L84F, A106V, D108N, J123Y, S146C, D147Y, R152P, E155V, I156F, and/or K157N.

15. The method of any one of claims 8-10, wherein the sgRNA-guided deactivated Cas9 and the adenine base editor (ABE) are separated by a linker.

16. The method of any one of claims 8-10, wherein the sgRNA-guided deactivated Cas9 that is fused to an adenine base editor (ABE) is further fused to a nuclear localization sequence (NLS), and/or an inhibitor of base repair, such as preferably a nuclease dead inosine specific nuclease (dISN).

17. The method of claim 12, wherein the viral vector is an adenovirus, adeno-associated virus, retrovirus, lentivirus, Newcastle disease virus (NDV), or lymphocytic choriomeningitis virus (LCMV).

18. The method of any one of claims 8-10, wherein the sgRNA-guided deactivated Cas9 that is fused to an adenine base editor is delivered to the subject via an exosome, lipid-based transfection, nanoparticle, or cell-based delivery system.

19. The method of any one of claims 1-17, wherein the CRISPR therapy results in cancer-cell senescence or reduced proliferation of the cancer.

20. The method of any one of claims 1-19, wherein the cancer is a glioblastoma, glioma, melanoma, hepatocellular carcinoma, urothelial carcinoma, medulloblastoma, squamous cell carcinoma such as of the tongue or head and neck, brain cancer, thyroid cancer, adrenal cortical carcinoma, tumors of the female reproductive organs, such as ovarian carcinoma, uterine clear cell carcinoma, cervical squamous cell carcinoma, mantle cell lymphoma, fibrosarcoma, myxoid liposarcoma, meningioma, or renal cell carcinoma.

21. The method of claim 20, wherein the cancer is a glioma, glioblastoma, or melanoma.

22. The method of any of claims 1-21, wherein the cancer contains a mutation in one or more oncogenes.

23. The method of claim 22, where the oncogene is K-Ras, B-Raf, EGFR, ALK, PI3K, BCR-ABL, IDH1, or IDH2.

24. The method of any one of claims 1-23, wherein the subject is a human.

25. A CRISPR therapy as described in any one of claims 1-24 for use in treating a cancer in a mammalian subject, preferably a human.

26. The CRISPR therapy of claim 25, wherein the cancer is a glioblastoma, glioma, melanoma, hepatocellular carcinoma, urothelial carcinoma, medulloblastoma, squamous cell carcinoma such as of the tongue or head and neck, brain cancer, thyroid cancer, adrenal cortical carcinoma, tumor of the a female reproductive organ, such as an ovarian carcinoma, uterine clear cell carcinoma, or cervical squamous cell carcinoma, mantle cell lymphoma, fibrosarcoma, myxoid liposarcoma, meningioma, or renal cell carcinoma.


SEQUENCE LISTING (41 sequences)

SEQ ID NO: 1 - protein, 167 aa, Artificial sequence
  Synthetic amino acid
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 2 - protein, 16 aa, Artificial sequence
  Synthetic amino acid
SGSETPGTSESATPES

SEQ ID NO: 3 - protein, 4 aa, Artificial sequence
  Synthetic amino acid
SGGS

SEQ ID NO: 4 - protein, 4 aa, Artificial sequence
  Synthetic amino acid
GGGS

SEQ ID NO: 5 - protein, 5 aa, Artificial sequence
  Synthetic amino acid
GGGGS

SEQ ID NO: 6 - protein, 5 aa, Artificial sequence
  Synthetic amino acid
EAAAK

SEQ ID NO: 7 - protein, 7 aa, Artificial sequence
  Synthetic amino acid
PKKKRKV

SEQ ID NO: 8 - protein, 30 aa, Artificial sequence
  Synthetic amino acid
MDSLLMNRRKFLYQFKNVRWAKGRRETYLC

SEQ ID NO: 9 - protein, 32 aa, Artificial sequence
  Synthetic amino acid
SGGSSGGSSGSETPGTSESATPESSGGSSGGS

SEQ ID NO: 10 - DNA, 100 bp, Artificial sequence
  Synthetic primer
cttctgttttagtccctgaagggactaaaataaagagtttgcgggactctgcggggttacaatcccctaaaaccgctttttttctagactgcagagggcc

SEQ ID NO: 11 - DNA, 100 bp, Artificial sequence
  Synthetic primer
ctctgcagtctagaaaaaaagcggttttaggggattgtaaccccgcagagtcccgcaaactctttattttagtcccttcagggactaaaacagaagagct

SEQ ID NO: 12 - DNA, 20 bp, Artificial sequence
  Synthetic primer
ggcccggaaggggctgggcc

SEQ ID NO: 13 - DNA, 20 bp, Artificial sequence
  Synthetic primer
gttccgcgttacataactta

SEQ ID NO: 14 - DNA, 20 bp, Artificial sequence
  Synthetic primer
cacatcatggcccctccctc

SEQ ID NO: 15 - DNA, 19 bp, Artificial sequence
  Synthetic primer
gaagccgaaggccagcacg

SEQ ID NO: 16 - DNA, 20 bp, Artificial sequence
  Synthetic primer
gagatcactgccctggcacc

SEQ ID NO: 17 - DNA, 19 bp, Artificial sequence
  Synthetic primer
gatggaggggccggactcg

SEQ ID NO: 18 - DNA, 22 bp, Artificial sequence
  Synthetic primer
caagttcctgcactggctgatg

SEQ ID NO: 19 - DNA, 23 bp, Artificial sequence
  Synthetic primer
caagtgctgtctgattccaatgc

SEQ ID NO: 20 - DNA, 20 bp, Artificial sequence
  Synthetic primer
ccttccagctccgcctcctc

SEQ ID NO: 21 - DNA, 19 bp, Artificial sequence
  Synthetic primer
cggggccgcggaaaggaag

SEQ ID NO: 22 - DNA, 63 bp, Artificial sequence
  Synthetic nucleic acid
ccagccccttccggcccagccccttccgggcccagccccctccgggcccagccccctccgggc

SEQ ID NO: 23 - DNA, 11 bp, Artificial sequence
  Synthetic nucleic acid
ccccttccggg

SEQ ID NO: 24 - DNA, 11 bp, Artificial sequence
  Synthetic nucleic acid
ggggaaggccc

SEQ ID NO: 25 - DNA, 11 bp, Artificial sequence
  Synthetic nucleic acid
ccccttccggg

SEQ ID NO: 26 - DNA, 9 bp, Artificial sequence
  Synthetic nucleic acid
ggggggccc

SEQ ID NO: 27 - DNA, 11 bp, Artificial sequence
  Synthetic nucleic acid
ccccccccggg

SEQ ID NO: 28 - DNA, 11 bp, Artificial sequence
  Synthetic nucleic acid
ggggggggccc

SEQ ID NO: 29 - DNA, 64 bp, Artificial sequence
  Synthetic nucleic acid
ccagccccttccgggcccagccccttccgggcccagccccttccgggcccagccccttccgggc

SEQ ID NO: 30 - DNA, 64 bp, Artificial sequence
  Synthetic nucleic acid
ccagccccttccgggcccagccccctccgggcccagccccctccgggcccagccccccccgggc

SEQ ID NO: 31 - DNA, 63 bp, Artificial sequence
  Synthetic nucleic acid
ccagccccttccgggcccagcccctccgggcccagccccttccgggcccagccccttccgggc

SEQ ID NO: 32 - DNA, 64 bp, Artificial sequence
  Synthetic nucleic acid
ccagccccttccgggcccagccccctccgggcccagccccctccgggcccagccccctccgggc

SEQ ID NO: 33 - DNA, 80 bp, Artificial sequence
  Synthetic nucleic acid
ccagccccttccgggcccagccccttccgggcccagccccttccgggcccagccccttccgggcccagccccttccgggc

SEQ ID NO: 34 - DNA, 64 bp, Artificial sequence
  Synthetic nucleic acid
ccagccccttccgggcccagccccttccgggcccagccccctccgggcccagccccctccgggc

SEQ ID NO: 35 - DNA, 64 bp, Artificial sequence
  Synthetic nucleic acid
ccagccccctccgggcccagccccctccgggcccagccccctccgggcccagccccctccgggc

SEQ ID NO: 36 - DNA, 32 bp, Artificial sequence
  Synthetic nucleic acid
ccagccccctccgggcccagccccctccgggc

SEQ ID NO: 37 - DNA, 32 bp, Artificial sequence; identical to SEQ ID NO: 36
  Synthetic nucleic acid

SEQ ID NO: 38 - DNA, 32 bp, Artificial sequence; identical to SEQ ID NO: 36
  Synthetic nucleic acid

SEQ ID NO: 39 - DNA, 32 bp, Artificial sequence; identical to SEQ ID NO: 36
  Synthetic nucleic acid

SEQ ID NO: 40 - DNA, 48 bp, Artificial sequence
  Synthetic nucleic acid
ccagccccttccgggcccagccccccccgggcccagccccccccgggc

SEQ ID NO: 41 - DNA, 48 bp, Artificial sequence
  Synthetic nucleic acid
ccagccccttccgggcccagccccctccgggcccagccccccccgggc